(19) United States
(12) Patent Application Publication
(10) Pub. No.: US 2024/0318226 A1
(43) Pub. Date: Sep. 26, 2024

(54) CIRCULATING TRANSCRIPTION FACTOR ANALYSIS

(71) Applicant: Belgian Volition SRL (BE/BE), Isnes (BE)

(72) Inventors: Jacob Vincent MICALLEF, London (GB); Mark Edward ECCLESTON, Cambridge (GB); Dorian Fernand François PAMART, Isnes (BE); Marielle HERZOG, Isnes (BE)

(21) Appl. No.: 18/260,018

(22) PCT Filed: Dec. 29, 2021
(86) PCT No.: PCT/EP2021/087813

§ 371 Date: Jun. 29, 2023

Related U.S. Application Data
(60) Provisional application No. 63/131,722, filed on Dec. 29, 2020.

(51) Int. Cl. C12Q 1/6804; C12Q 1/6806; C12Q 1/6886

(52) U.S. Cl. CPC C12Q 1/6804; C12Q 1/6806; C12Q 1/6886; G01N 2800/52

(57) ABSTRACT

The invention relates to methods for detecting disease in a subject by means of a minimally invasive body fluid test for the detection of circulating chromatin fragments that include a transcription factor and associated DNA sequence as an indicator of the presence of disease in the subject, e.g. cancer. The methods may comprise sequencing the associated DNA sequence and/or removing cell free nucleosomes from the body fluid.

### FIELD OF THE INVENTION

The invention relates to a method for detecting disease in a subject by means of a minimally invasive body fluid test. The invention also relates to the measurement or detection of circulating chromatin fragments that include a transcription factor as an indicator of the presence of disease in the subject.

### BACKGROUND OF THE INVENTION

Cancer is a common disease with a high mortality. The biology of the disease is understood to involve a progression from a pre-cancerous state leading to stage I, II, III and eventually stage IV cancer. For the majority of cancer diseases, mortality varies greatly depending on whether the disease is detected at an early localized stage, when effective treatment options are available, or at a late stage when the disease may have spread within the organ affected or beyond when treatment is more difficult. Late stage cancer symptoms are varied including visible blood in the stool, blood in the urine, blood discharged with coughing, blood discharged from the vagina, unexplained weight loss, persistent unexplained lumps (e.g. in the breast), indigestion, difficulty in swallowing, changes to warts or moles as well as many other possible symptoms depending on the cancer type. However, most cancers diagnosed due to such symptoms will already be late stage and difficult to treat. Most cancers are symptomless at early stage or present with non-specific symptoms that do not help diagnosis. Cancer should ideally therefore be detected early using cancer tests.

To address the need for simple routine cancer blood tests, many blood borne proteins have been investigated as potential cancer biomarkers including carcinoembryonic antigen (CEA) for CRC, alpha-fetoprotein (AFP) for liver cancer, CA125 for ovarian cancer, CA19-9 for pancreatic cancer, CA15-3 for breast cancer and PSA for prostate cancer. However, their clinical accuracy is too low for routine diagnostic use and they are considered to be better used for patient monitoring.

More recently, workers in the field have investigated circulating tumor DNA (ctDNA) as a blood based biomarker for cancer detection. Cell free DNA (cfDNA) circulates in the blood as chromatin fragments that are thought to originate from cell death, mainly by apoptosis, of a huge number of cells daily. During the process of apoptosis chromatin is fragmented into mononucleosomes and oligonucleosomes, some of which are released from the cells to circulate as cell free nucleosomes. Each circulating cell free nucleosome is associated with a small DNA fragment of less than 200 base pairs (bp) in length. Similarly, cell free chromatin fragments consisting of DNA bound transcription factors, or other non-histone chromatin proteins, in the circulation has been inferred from fragmentomics analysis. In healthy subjects circulating chromatin fragments are thought to be of hematopoietic origin and levels are low. Elevated levels of circulating nucleosomes, and hence cfDNA fragments, are found in subjects with a variety of conditions including many cancers, auto-immune diseases, inflammatory conditions, stroke and myocardial infarction (Holdenrieder & Stieber, 2009).

At least some of the cfDNA in the blood of cancer patients is thought to originate from the release of nucleosomes and other chromatin fragments into the circulation from dying or dead cancer cells (i.e. the cfDNA includes some ctDNA). Investigation of matched blood and tissue samples from cancer patients shows that cancer associated mutations, present in a patient's tumor (but not in his/her healthy cells) are also present in cfDNA in blood samples taken from the same patient (Newman et al, 2014). Similarly, DNA sequences that are differentially methylated (epigenetically altered by methylation of cytosine residues) in cancer cells can also be detected as methylated sequences in cfDNA in the circulation. In addition, the proportion of circulating cfDNA that is comprised of ctDNA is related to tumor burden so disease progression may be monitored both quantitatively by the proportion of ctDNA present and qualitatively by its genetic and/or epigenetic composition. Analysis of ctDNA can produce highly useful and clinically accurate data pertaining to DNA originating from all or many different clones within the tumor and which hence integrates the tumor clones spatially. Moreover, repeated blood sampling over time is a much more practical and economic option than, for example, repeated tissue biopsy. Analysis of ctDNA has the potential to revolutionize the detection and monitoring of tumors, as well as the detection of relapse and acquired drug resistance at an early stage for selection of treatments for tumors through the investigation of tumor DNA without invasive tissue biopsy procedures. Such ctDNA tests may be used to investigate all types of cancer associated DNA abnormalities (e.g. point mutations, nucleotide modification status, translocations, gene copy number, micro-satellite abnormalities and DNA strand integrity) and would have applicability for routine cancer screening, regular and more frequent monitoring and regular checking of optimal treatment regimens (Zhou et al, 2017).

Blood plasma is commonly used as substrate for ctDNA assays. The cfDNA fragments (including any ctDNA) are extracted from the plasma (and hence removed from binding to nucleosomes, transcription factors or other proteins) and analyzed for nucleotide base sequence. Any DNA analysis method may be employed but typically analysis is performed by deep sequencing using Next Generation Sequencer instrumentation.

As DNA abnormalities are characteristic of all cancer diseases and ctDNA has been observed for all cancer diseases in which it has been investigated, ctDNA tests have applicability in all cancer diseases. Cancers investigated include, without limitation, cancer of the bladder, breast, colorectal, melanoma, ovary, prostate, lung liver, endometrial, ovarian, lymphoma, oral, leukaemias, head and neck, and osteosarcoma (Crowley et al, 2013; Zhou et al, 2017; Jung et al, 2010).

One example method of cfDNA analysis involves the identification of the tissue or cells of origin of the cfDNA fragments of a subject. The basis of this approach is that all cfDNA fragments present in the circulation have avoided digestion by nucleases during cell death or in the circulation because they are protected from nuclease action by protein binding within nucleosomes. The approach involves the determination of the nucleosome fragmentation pattern of cfDNA in a blood sample taken from the subject and locating the genomic position of the cfDNA fragments in a reference genome. The pattern of fragmentation differs for different cell types and can be used to identify the cells of origin of the cfDNA of the subject.

This approach involves extraction of cfDNA (including any ctDNA) from a plasma sample and whole genome sequencing of the DNA to detect the nucleosome bound DNA pattern displayed by the cfDNA fragments. The endpoint sequences of the cfDNA fragments are located for their genomic position within a reference genome or genomes using bioinformatics by computer analysis. The genomic locations of the cfDNA endpoints within the reference genome provides a map of the nucleosome protected cfDNA coverage of the genome.

The proportional contributions of different cell types or tissues to the cfDNA in a subject may also be determined by comparison of the nucleosome fragmentation patterns of the subject to calibration samples containing known relative abundance of cfDNA from different cellular sources using bioinformatics by computer analysis as described in WO2017012592.

The cfDNA fragments associated with chromatin fragments containing nucleosomes are typically 120-200 bp in length. However, protein binding and protection of cfDNA is not limited to the histone binding of cfDNA in nucleosomes. Other cfDNA fragments, including active gene promoter sequences, are bound by transcription factors, cofactors or other non-histone chromatin proteins either in addition to a nucleosome or in the absence of any nucleosome. In the absence of a nucleosome, these proteins often bind and protect shorter cfDNA fragments in the range of 35-80 bp. However, these shorter cfDNA fragments are only observed experimentally if the DNA fragment library preparation method used is suitable for the isolation, amplification and sequencing of short DNA fragments of less than 100 base pairs in length (Snyder et al, 2016).

The pattern of protein binding of DNA across the genome in living cells varies with cell type because different DNA sequences, including different promoter sequences and genes sequences, are active in different cells. The pattern of protein binding of DNA in any cell type can be determined by Nuclease Accessible Site mapping by digestion of chromatin extracted from the cell with a nuclease enzyme and sequencing the undigested DNA in the resulting protein-protected chromatin fragments. Thus, if one views the cfDNA fragments in the blood as the product of an in vivo nuclease digestion, the cfDNA sequences found should correspond to protein bound DNA sequences in the cell from which the cfDNA originated. In principle, therefore, the pattern of cfDNA fragment sequences in the blood should be similar to the pattern of sequences of chromatin fragments generated by Nuclease Accessible Site mapping of the cells of origin. Thus, the fragmentation pattern of cfDNA sequences determined from a blood sample can be compared using bioinformatics methods to known DNA fragmentation patterns generated by Nuclease Accessible Site analysis of cells of known tissue or cancer type to determine the tissue of origin of the cfDNA. The results in samples taken from healthy subjects indicate that the cells of origin of cfDNA are hematopoietic. The results of this approach in samples taken from cancer patients indicate that the cfDNA and ctDNA originate from a mixture of cells including hematopoietic cells and other cells. In many cases the non-hematopoietic cell type indicated correlates with the tissue of the cancer disease of the patient (Snyder et al, 2016).

Other workers have used a similar cfDNA fragment endpoint analysis approach involving whole genome cfDNA sequencing (including any ctDNA), but focused the bioinformatic computer analysis on transcription factor binding site (TFBS) sequences. The aim of this approach is to determine TFBS accessibility and identify TFBS DNA sequences with altered accessibility in plasma samples taken from patients with cancer (Ulz et al, 2019). In this approach, a blood plasma sample is taken from a subject and the cfDNA is extracted and amplified using a DNA library preparation method suitable for small DNA fragments of less than 100 bp in length. The DNA library is sequenced using a next generation sequencing method. The sequencing data is used to identify the cfDNA fragmentation pattern in the genomic region near to a TFBS using bioinformatics methods. The analysis involves determining the nucleosome positioning profile of cfDNA fragments across a TFBS and its flanking sequences in a gene promoter sequence to determine whether or not the TFBS was bound to a transcription factor in the chromatin fragments that comprised the cfDNA. The method is complex but can be summarized as follows:

If the cfDNA fragmentation pattern observed in the DNA sequences that span a TFBS and flanking sequences in the genome displays a periodicity of approximately 200 bp, this relates to alternating stronger protein binding protection (at the center of a nucleosome binding position) and weaker protein binding protection (between nucleosomes where the DNA is unbound and unprotected) of DNA from degradation. In this case, the TFBS and flanking sequences is assumed to have been nucleosome covered in the chromatin fragments that comprised the cfDNA in the plasma sample.

If the cfDNA fragmentation pattern present additionally displays protein binding protection of a TFBS and its flanking sequences, but with no (or an attenuated) nucleosome related periodicity, this relates to transcription regulatory protein binding at the TFBS and its flanking sequences. In this case, the TFBS is assumed to have been bound to one or more transcription factors and/or other regulatory proteins in the chromatin fragments that comprised the cfDNA in the plasma sample.

In healthy subjects, the cfDNA fragmentation pattern found typically correlates with the pattern obtained for nuclease accessible site experiments of haemopoietic cells. Thus, the TFBS sequences that are transcription factor bound or nucleosome covered in the cfDNA correlate with transcription factors that are, or are not, expressed in haemopoietic cells. In cancer patients, the pattern relates to a mixture of cell types in which the TFBS may be transcription factor bound in the cancer cell type and nucleosome bound in hematopoietic cell type. As most cfDNA is derived from hematopoietic cells and only a small amount is derived from cancer cells, the cancer derived fragmentomics signal is small compared to the hematopoietic signal. However, fragmentomics bioinformatics methods have been developed to disentangle the small transcription factor protected TFBS fragment signal present in ctDNA from the much greater superimposed nucleosome periodicity signal present in the hematopoietic derived cfDNA component. Fragmentomics analysis indicates that the mixed pattern includes cfDNA TFBS sequences that are transcription factor bound for transcription factors that are not expressed in haemopoietic cells, but expressed by the cancer tissue.

Chromatin Immunoprecipitation followed by sequencing of the chromatin associated DNA (ChIP-Seq) is an analytical technique used to map the genomic location of cellular chromatin proteins. A typical method involves extraction of chromatin from a cell followed by digestion of the chromatin into mononucleosomes or other chromatin fragments by physical disruption (for example, sonication) or by using a nuclease enzyme that cleaves DNA (for example, DNase or Micrococcal Nuclease). The fragmented chromatin is then exposed to a solid phase support coated with an antibody directed to bind to a particular chromatin protein of interest, for example a particular modified histone. Chromatin fragments comprising the particular structure are adsorbed (immunoprecipitated) onto the solid phase. DNA associated with the adsorbed chromatin is then extracted from the solid phase and amplified by a polymerase chain reaction (PCR) method. The amplified DNA fragment library is sequenced to determine the locations within the genome where the chromatin protein of interest was bound. ChIP methods using antibodies to transcription factors are also used to identify the genomic locations of Transcription Factor Binding Sites (TFBS) of a particular transcription factor or whether or not a particular TFBS is occupied by a particular transcription factor in different cell types.

We have previously described immunoassay tests for circulating cell free nucleosomes containing particular epigenetic signals including particular post-translational modifications, histone isoforms, modified nucleotides and non-histone chromatin proteins for the detection of cancer and other diseases (as referenced in WO2005019826, WO2013030577, WO2013030579 and WO2013084002). We have also described immunoassay tests for chromatin fragments including transcription factor bound DNA for the detection of cancer (as referenced in WO2017162755).

We now report methods with superior analytical and clinical specificity and sensitivity for the isolation and direct analysis and measurement of circulating cell free chromatin fragments containing one or more transcription factors together with an associated fragment of DNA. Isolation of transcription factor-DNA complexes from the much more numerous nucleosome fragments simplifies the analysis and eliminates the need to disentangle transcription factor covered TFBS signals from dominant nucleosome periodicity signals. The methods may be used in blood samples as non-invasive, or minimally invasive, blood tests for diseases including cancer, autoimmune diseases and inflammatory diseases.

### SUMMARY OF THE INVENTION

According to a first aspect of the invention there is provided a method of detecting a cell free chromatin fragment comprising a transcription factor and a DNA fragment in a body fluid sample obtained from a human or animal subject, which comprises the steps of:
(i) contacting the body fluid sample with a binding agent which binds to the transcription factor;
(ii) detecting or measuring the DNA fragment associated with the transcription factor; and
(iii) using the presence or amount of the DNA fragment as a measure of the amount of cell free chromatin fragments comprising the transcription factor in the sample.

According to a further aspect of the invention, there is provided a method of detecting a disease in a human or animal subject which comprises the steps of:
(i) contacting a body fluid sample obtained from the human or animal subject with a binding agent which binds to a transcription factor;
(ii) detecting or measuring the DNA associated with the transcription factor; and
(iii) using the presence or amount of DNA as an indicator of the presence of a disease in the subject.

According to a further aspect of the invention, there is provided a method of detecting a tissue affected by a disease in a human or animal subject which comprises the steps of:
(i) contacting a body fluid sample obtained from the human or animal subject with a binding agent which binds to a transcription factor;
(ii) sequencing the DNA associated with the transcription factor; and
(iii) using the presence of the transcription factor and the sequence of the associated DNA as a combined biomarker for determining the tissue affected by the disease in the subject.

According to a further aspect of the invention, there is provided a method for the assessment of an animal or a human subject for suitability for a medical treatment which comprises the steps of:
(i) detecting, measuring or sequencing DNA associated with a cell free chromatin fragment comprising a transcription factor in a body fluid sample obtained from the subject; and
(ii) using the associated DNA level and/or sequence detected in step (i) as a parameter for the selection of a suitable treatment for the subject.

According to a further aspect of the invention, there is provided a method for monitoring a treatment of an animal or a human subject which comprises the steps of:
(i) detecting, measuring or sequencing DNA associated with a cell free chromatin fragment comprising a transcription factor in a body fluid sample obtained from the subject;
(ii) repeating the detection, measurement or sequencing of DNA associated with a cell free chromatin fragment comprising the transcription factor in a body fluid sample obtained from the subject on one or more occasions; and
(iii) using any changes in the associated DNA level and/or DNA sequence detected in step (i) compared to step (ii) as a parameter for any changes in the condition of the subject.

According to a further aspect of the invention, there is provided a kit for the detection of a cell free chromatin fragment comprising a transcription factor and a DNA fragment as a combination biomarker which comprises a ligand or binder for the transcription factor optionally together with reagents for the amplification and or sequencing of DNA associated with said transcription factor, and/or a ligand or binder for nucleosomes and/or instructions for use of the kit in accordance with the method as defined herein.

According to a further aspect of the invention, there is provided a method of treating cancer in a subject in need thereof, wherein said method comprises the following steps:
(a) contacting a body fluid sample obtained from the human or animal subject with a binding agent which binds to a transcription factor;
(b) detecting, measuring or sequencing a DNA fragment associated with the transcription factor; and
(c) using the presence or amount of DNA fragment as an indicator of the presence of cancer in the subject; and
(d) administering a treatment if the subject is determined to have cancer in step (c).

A method of detecting a disease in a human or animal fetus which comprises the steps of:
(i) obtaining a body fluid sample from a pregnant human or animal subject;
(ii) contacting the body fluid sample with a binding agent which binds to a transcription factor;
(iii) detecting, measuring or sequencing the DNA associated with the transcription factor; and
(iv) using the presence, sequence or amount of DNA as an indicator of the presence of a disease in the fetus.

### BRIEF DESCRIPTION OF THE FIGURES

FIG. 1: A cartoon illustration of the co-binding of various transcription factors at the promoter sites of the surfactant protein B, thyroglobulin, thyroperoxidase and thyrotropin receptor (TSH receptor) genes. CRE: cyclic adenosine monophosphate response element; GABP: GA-binding protein; HNF-3: Hepatocyte nuclear factor 3; NF-1: Nuclear factor 1; PAX-8: Paired box gene 8; Runx2: Runt-related transcription factor 2; TRα/RXR dimer: Thyroid hormone receptor a/Retinoid X receptor dimer; TTF-1: Thyroid transcription factor 1 (also known as NK2 homeobox 1, NKX2-1); TTF-2: Thyroid transcription factor 2.

FIG. 2: A cartoon of an example of the DNA loop structure of a transcription complex, to illustrate co-binding of some of the various regulatory proteins involved in a transcription complex including, without limitation, general transcription factors (GTF), gene specific transcription factors (TF), co-factors, activators, repressors, mediators, DNA bending proteins and RNA Polymerase. The regulatory proteins are bound to regulatory DNA sequences located near to the gene as well regulatory sequences far from the gene, including promoter sequences, TATA box sequences, enhancer sequences and repressor sequences. Other regulatory proteins (for example chromatin remodeling proteins) as well as other regulatory sequences are possible.

FIG. 3: Western blot analysis of recombinant mononucleosomes adsorbed onto magnetic beads coated with an antibody directed to bind to histone H3. The results demonstrate dose dependent adsorption of mononucleosomes.

FIG. 4: Nucleosome ELISA results for human plasma samples and solutions of recombinant mononucleosomes following immunoprecipitation of nucleosomes using uncoated magnetic beads or magnetic beads coated with an antibody directed to bind to histone H3. The results demonstrate that both naturally occurring human circulating nucleosomes and recombinant nucleosomes in solution were unaffected by uncoated magnetic beads but were quantitatively removed by immunoprecipitation using magnetic beads coated with an antibody directed to bind to histone H3.

FIG. 5: Levels of ERα measured in women diagnosed with ER-negative breast cancer (ER-BC), ovarian cancer or ER-positive breast cancer (ER+BC) with an ER score of 7 or 8.

FIG. 6: The effect of washing magnetic polystyrene particles exposed to a plasma sample obtained from a cancer patient with a regular single detergent wash buffer containing 0.1% Tween (0.1%) or with a strong wash buffer containing a mixture of detergents totaling 1.2% detergent (1.2%). The non-specific IgG coated particles showed a greater reduction in background binding through use of a strong detergent wash (lanes 4 and 5) without disruption of specific antibody bound proteins (a mixture of parylated proteins) (lanes 6 and 7).

FIG. 7: Western Blot analysis of chromatin fragments immunoprecipitated from 4 pooled cross-linked EDTA plasma samples taken from patients diagnosed with CRC by ChIP using a mouse anti-CTCF antibody immobilized on magnetic polystyrene beads washed using the strong 1.2% detergent mix wash buffer. All 4 plasma samples showed a band at around 140 kD corresponding to CTCF protein (Anti CTCF; lanes 3, 5, 7 and 9). Negative control experiments using non-specific mouse IgG showed no band corresponding to CTCF (NS-IgG; lanes 2, 4, 6 and 8). The experiment demonstrated that CTCF protein was isolated from the plasma samples and that use of a strong wash buffer led to a relatively pure CTCF extract from plasma.

FIG. 8: Electropherograms showing analysis of the amplified adapter ligated cfDNA fragment library resulting from ChIP of CTCF chromatin fragments in a cross-linked EDTA plasma sample taken from a patient. The sharp peak at approximately 140 bp represents the adapter dimer, so adapter linked fragments of 175-220 bp represent cfDNA fragments of 35-80 bp (indicated on electropherograms). (a) The specific CTCF ChIP library contained small cfDNA fragments with a fluorescence peak of approximately 1000 FU in the range of 35-80 bp. (b) The non-specific control IgG library also contained small cfDNA fragments with a fluorescence peak of approximately 80 FU.

FIG. 9: Normalised coverage of 9780 published CTCF TFBS loci by transcription factor bound (35-80 bp) or nucleosome bound (135-155 bp or 156-180 bp) cfDNA fragments. (a) Specific CTCF coverage by a cfDNA sequence library obtained for a CRC patient. (b) Non-specific coverage by a cfDNA sequence library obtained from chromatin fragments bound non-specifically to mouse IgG coated particles. The results show that the peak of specific cfDNA coverage originating from plasma circulating CTCF-DNA complexes correlates with published CTCF TFBS loci. The expected oscillating coverage pattern due to nucleosome binding is minimal across the 5 kb span investigated. In the control sample no peak cfDNA coverage at the CTCF binding loci was observed.

FIG. 10: Normalised coverage of 1041 published CTCF TFBS loci occupied by CTCF in cancer cells but not in normal cells. Coverage is shown for transcription factor bound (35-80 bp) or nucleosome bound (135-155 bp or 156-180 bp) cfDNA fragments. (a) CTCF occupation of cancer associated loci by a cfDNA sequence library obtained for a CRC patient. The results show coverage in the 35-80 bp size range confirming CTCF occupancy of some or all of these 1041 sites and are therefore indicative of cancer in the subject from whom the sample was taken. (b) There was no CTCF occupancy peak observed in a non-specific control experiment.

FIG. 11: Western Blot analysis of chromatin fragments immunoprecipitated from 8 cross-linked EDTA plasma samples by ChIP using a mouse anti-AR antibody immobilized on magnetic polystyrene beads washed using the strong 1.2% detergent mix wash buffer. All 8 plasma samples (S1-S8; lanes 2-9) showed a band at around 140 kD corresponding to AR protein. The highest density bands were observed for samples S1 and S2. Lane 10 represents a positive control using fragmented chromatin from LnCAP prostate cancer cells.

FIG. 12: Electropherograms showing analysis of the amplified adapter ligated cfDNA fragment library resulting from ChIP of AR chromatin fragments in cross-linked EDTA plasma samples taken from 8 prostate cancer patients (S1-S8). The sharp peak at approximately 140 bp represents the adapter dimer, so adapter linked fragments of 175-220 bp represent cfDNA fragments of 35-80 bp. An electropherogram for a negative control (ctrl) is also shown.

### DETAILED DESCRIPTION OF THE INVENTION

Transcription factors are involved in cancer and account for about 20% of all known oncogenes (Lambert et al, 2018). We have previously described the use of a chromatin fragment containing a tissue specific transcription factor as a biomarker in serum for the detection or diagnosis of a cancer in a subject. The tissue specificity of the transcription factor can be used to indicate the tissue of origin of a cancer. For example, the transcription factor TTF-1 is reported to be expressed in thyroid and lung tissue and not in other tissues. The presence of circulating chromatin fragments containing TTF-1 therefore indicates the tissue of origin is lung or thyroid. We also described immunoassay methods for the measurement of circulating cell free chromatin fragments containing transcription factors. This immunoassay involves a double-antibody (or other binder) method where one antibody is directed to bind to a transcription factor and the other to bind to DNA associated with the transcription factor or to a nucleosome component included in a chromatin fragment. In one embodiment described, the binder targeted to bind to a transcription factor is immobilized on a solid phase to isolate the chromatin fragment containing the transcription factor (i.e. to immunoprecipitate the chromatin fragment). The isolated chromatin fragment is then detected using a second binder directed to bind to DNA. This immunoassay method is simple, low cost and non-invasive.

ChIP-Seq is a method normally applied to cellular chromatin extracts following fragmentation by enzyme digestion with a nuclease or by sonication. There are a few reports of the application of ChIP-Seq methods in EDTA plasma. As chromatin in plasma is already fragmented, nuclease digestion or sonication of the sample is not required. Reports of ChIP-Seq in plasma relate to the isolation of histone proteins from EDTA plasma using anti-histone antibodies followed by extraction, amplification and sequencing of the histone associated DNA fragments (Deligezer et al, 2008, Mansson et al, 2021, Sadeh et al, 2021, Vad-Nielsen et al, 2020).

To the authors' knowledge, there are no ChIP-Seq methods described in the literature for the direct isolation, analysis or mapping of intact circulating transcription factor-DNA chromatin fragments and associated TFBS DNA sequences. Instead, workers in the field have developed indirect methods based on the analysis of DNA fragments.

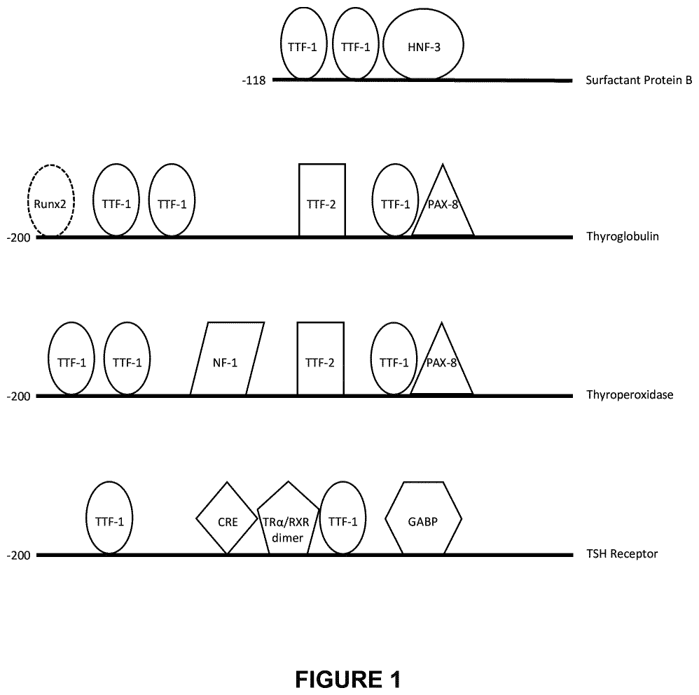

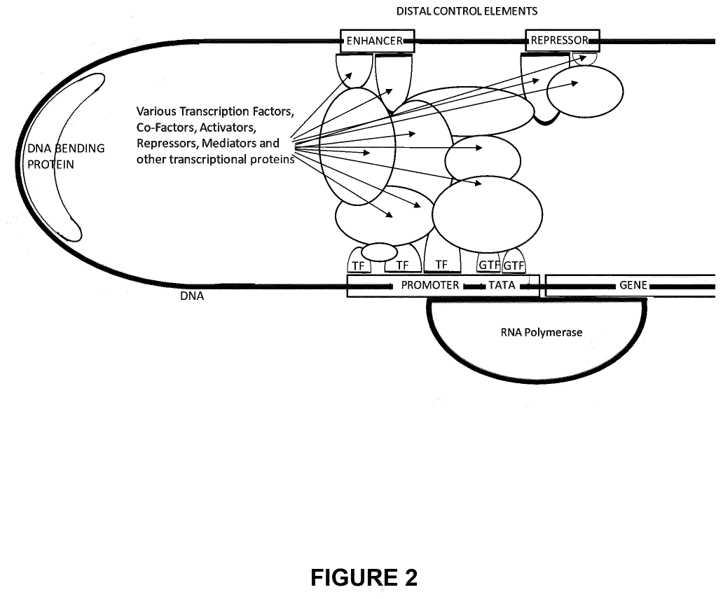

Fragmentomics is one such indirect method in which deep sequencing of cfDNA extracted from EDTA plasma are analysed by bioinformatics methods to identify DNA fragmentation patterns that are indicative of transcription factor-DNA binding in the original sample (Snyder et al, 2016, Ulz et al, 2019). This is an indirect method because the first step in fragmentomics is the extraction of all DNA in the sample investigated and this necessarily involves the destruction of all transcription factor-DNA complexes present. This destroys all information directly linking any DNA fragment or sequence with any transcription factor or other chromatin protein in the sample. The occupancy of a TFBS is inferred from the presence of short cfDNA fragments (35-80 bp) of an appropriate sequence in the extracted DNA library. However, the identity of the chromatin protein that was attached to the DNA fragment (prior to DNA extraction) cannot be known, particularly as many proteins may be bound in close proximity to the site of interest as shown in FIGS. 1 and 2. One disadvantage of fragmentomics methods is therefore that the binding of any particular transcription factor at any particular TFBS may be inferred but cannot be established.

Another recent indirect method involves nucleosome ChIP-Seq in EDTA plasma to directly map cell-free nucleosome positioning and using nucleosome positioning data to indirectly infer transcription factor positioning (Sadeh et al, 2021).

The reason direct ChIP methods for transcription factor-DNA complexes have not been reported is that there are significant technical difficulties or hurdles that have hitherto not been addressed. These technical difficulties include (i) recognition that some transcription factor-DNA complexes are stably associated in plasma whilst other transcription factor-DNA complexes that are dynamically associated in vivo will be disassociated in blood or other body fluids, (ii) recognition that the most common class of transcription factor-DNA complexes are disassociated in EDTA plasma but this can be prevented, (iii) nuclear extracts from cellular or tissue material are relatively pure chromatin preparations that can be obtained in μg or mg amounts. In contrast blood, serum or plasma contains very low levels of very impure chromatin “contaminated” with high levels of other circulating proteins, (iv) there are at least many hundreds of transcription factors and any particular transcription factor-DNA complex will be only one of many thousands of different transcription factor-DNA complexes present in the plasma. In turn the total transcription factor-DNA fraction of cfDNA is a small fraction of total cfDNA (most of which comprises nucleosome fragments) and the proportion of cfDNA originating from cancer cells is a small fraction of total cfDNA. Transcription factor-DNA complexes including any particular transcription factor are therefore a small fraction of a small fraction of a small fraction contaminated with high levels of other proteins and other substances. One consequence of this is that the specific signal generated in a plasma transcription factor-DNA ChIP-Seq method will be small (smaller than the background signal) making effective data analysis problematic.

We now report methods for the detection of circulating cell free chromatin fragments containing a transcription factor-DNA complex with superior analytical sensitivity and superior tissue specificity. The methods also widen the use of applicable transcription factors to include most or all transcription factors.

We also report the use of a combination biomarker consisting of a chromatin fragment containing a transcription factor in combination with the sequence of the DNA fragment associated with said transcription factor for the detection of disease. This combination biomarker additionally has very high tissue specificity and can be used as biomarker of cancer.

Analytical sensitivity is important for circulating cell free chromatin fragments containing transcription factors that occur at low levels, near to, or below, the limits of detection by immunoassay. The analytical limit of detection of immunoassays varies with the design of the assay and with the affinity of the binder used (usually an antibody) but may be in the picomolar concentration range. However, the analytical sensitivity of the polymerase chain reaction (PCR) detection of DNA is orders of magnitude lower. Digital PCR may detect concentrations as low as a few individual molecules per sample. Therefore, use of a PCR amplification method for the detection of the DNA associated with a transcription factor, rather than by use of an antibody directed to bind to DNA (or to a nucleosome epitope) allows the detection of circulating chromatin fragments containing a transcription factor at extremely low levels.

As well as increased sensitivity through use of PCR for detection, analysis of chromatin fragments containing transcription factors based on their associated DNA content also leads to high analytical sensitivity by addressing the large pool of transcription factors that do not comprise an associated nucleosome.

Therefore, according to a first aspect of the invention, there is provided a method of detecting a cell free chromatin fragment comprising a transcription factor and a DNA fragment in a body fluid sample obtained from a human or animal subject which comprises the steps of:
(i) contacting the body fluid sample with a binding agent which binds to a transcription factor;
(ii) detecting or measuring the DNA fragment associated with the transcription factor; and
(iii) using the presence or amount of the DNA fragment as a measure of the amount of cell free chromatin fragments comprising the transcription factor in the sample.

In one embodiment, the antibody or other binder of a transcription factor used in step (i) is immobilized on a solid phase to isolate the transcription factor from the sample.

In one embodiment, the method comprises isolating the transcription factor bound in step (i), i.e. from the remaining body fluid sample, prior to detection of the associated DNA fragment. For example, a wash buffer may be applied to the transcription factors in the sample bound to the (solid phase) binding agent in step (i) to remove the remaining sample which is not bound to the binding agent.

In one embodiment, transcription factor associated DNA fragments are extracted from the transcription factor for detecting, measuring or sequencing the DNA fragment in step (ii).

In one embodiment the DNA is detected or measured using a general DNA binder such as an anti-DNA antibody or a DNA chelating or intercalating agents, for example, ethidium bromide and cyanine dyes such as SYBR green and SYBR gold.

In one embodiment, step (ii) comprises sequencing the DNA fragment associated with the transcription factor. Sequencing methods are well known in the art.

According to some embodiments, detecting or measuring the DNA fragment in step (ii) is performed by amplification of the DNA fragment, for example using quantitative PCR method to determine the presence and/or amount of DNA fragment. Therefore, according to a further aspect of the invention there is provided a method of detecting a cell free chromatin fragment comprising a transcription factor and a DNA fragment in a human or animal subject which comprises the steps of:
(i) contacting a body fluid sample obtained from the human or animal subject with a binding agent which binds to a transcription factor;
(ii) isolating the DNA associated with the transcription factor;
(iii) amplifying the DNA; and
(iv) using the presence or amount of the DNA fragment as a measure of the presence or amount of cell free chromatin fragments comprising the transcription factor in the sample.

In one embodiment the amplified DNA is detected or measured using a DNA hybridization method.

In a further embodiment, amplification of the transcription factor bound DNA fragment is performed following ligation of adapter oligonucleotides to the DNA fragment. Adapter oligonucleotides may include primer sequences to facilitate amplification of DNA fragments by PCR or primer sequences may be added subsequently. Methods involving adapter oligonucleotides are well known in the art and routinely used to prepare libraries for next generation sequencing. Therefore, in one embodiment of the invention there is provided a method of detecting a cell free chromatin fragment comprising a transcription factor and a DNA fragment in a human or animal subject which comprises the steps of:
(i) contacting a body fluid sample obtained from the human or animal subject with a binding agent which binds to a transcription factor;
(ii) isolating the DNA associated with the transcription factor;
(iii) ligating an adapter oligonucleotide to the isolated DNA;
(iv) amplifying the DNA; and
(v) using the presence or amount of the DNA fragment as a measure of the amount of cell free chromatin fragments comprising the transcription factor in the sample.

In one embodiment, amplification of the transcription factor bound DNA fragment is performed using PCR primer oligonucleotides of specific sequence(s) designed for the amplification of DNA fragments including particular sequence(s). This embodiment facilitates the amplification of selected DNA fragments including the TFBS sequence(s) and/or flanking sequence(s). This embodiment is also rapid, low cost, easily automated for high throughput, may be performed in any PCR laboratory and additionally further increases the healthy or diseased cfDNA tissue of origin specificity by combining the joint tissue specificity of transcription factor expression with the specificity of identifying the location of its binding in the genome through analysis of the TFBS sequence and/or flanking sequences of the associated DNA in the chromatin fragment. Therefore, in one embodiment of the invention there is provided a method of detecting a cell free chromatin fragment comprising a transcription factor and a DNA fragment in a human or animal subject which comprises the steps of:
(i) contacting a body fluid sample obtained from the human or animal subject with a binding agent which binds to a transcription factor;
(ii) isolating the DNA associated with the transcription factor;
(iii) amplifying the DNA using a sequence specific PCR primer oligonucleotide; and
(iv) using the presence or amount of the DNA fragment as a measure of the amount of cell free chromatin fragments comprising the transcription factor in the sample.

In one embodiment the method comprises extracting the DNA fragment associated with the transcription factor. In a further embodiment, the method comprises amplification of the extracted DNA fragment. Therefore, according to a further aspect of the invention, there is provided a method of detecting a cell free chromatin fragment comprising a transcription factor and a DNA fragment in a body fluid sample obtained from a human or animal subject which comprises the steps of:
(i) contacting the sample with a binding agent which binds to a transcription factor;
(ii) isolating the bound transcription factor;
(iii) extracting the DNA associated with the transcription factor;
(iv) amplifying the extracted DNA;
(v) detecting the amplified extracted DNA; and
(vi) using the presence or amount of DNA as a measure of the amount of cell free chromatin fragments comprising the transcription factor in the sample.

In preferred embodiments, the amplification of the transcription factor associated DNA is performed by PCR. There are many PCR methods known in the art including, without limitation, quantitative PCR, real time PCR, reverse transcriptase PCR, nested PCR, digital PCR, multiplex PCR, arbitrary primed PCR, cold PCR (co-amplification at lower denaturation temperature-PCR). In some embodiments the amplification method includes DNA quantification.

According to a further aspect of the invention, there is provided a method of detecting a disease in a human or animal subject which comprises the steps of:
(i) contacting a body fluid sample obtained from the human or animal subject with a binding agent which binds to a transcription factor;
(ii) detecting or measuring the DNA associated with the transcription factor; and
(iii) using the presence or amount of DNA as an indicator of the presence of a disease in the subject.

In another aspect of the invention, there is provided a method of detecting a disease in a human or animal subject which comprises the steps of:
(i) contacting a body fluid sample obtained from the human or animal subject with a binding agent which binds to a transcription factor;
(ii) isolating the DNA associated with the transcription factor;
(iii) contacting the DNA with a DNA binding agent;
(iv) detecting the DNA binding agent; and
(v) using the presence or amount of DNA binding agent as an indicator of the presence and/or the nature of a disease in the subject.

Any DNA binding agent may be suitable for use in the invention including antibodies. The DNA binding agent may be directly or indirectly (for example, through a linker system such biotin/avidin or glutathione) labelled with a detectable moiety such as a fluorescent, enzymic or radioactive moiety.

In another aspect of the invention there is provided a method for determining the genomic TFBS locations occupied by a particular transcription factor (and hence also which genes were being regulated) by detecting a cell free chromatin fragment comprising a transcription factor and an associated fragment of DNA wherein the DNA fragment associated with a transcription factor is sequenced to determine the genomic location at which the transcription factor was bound. Therefore, in another aspect of the invention, there is provided a method for determining the genomic location where a transcription factor binds which comprises the steps of:
(i) contacting a sample with a binding agent which binds to the transcription factor;
(ii) isolating the bound transcription factor;
(iii) extracting the DNA associated with the transcription factor;
(iv) amplifying the extracted DNA;
(v) sequencing the amplified extracted DNA; and
(vi) using the sequence of the extracted DNA to determine the genomic location of the TFBS.

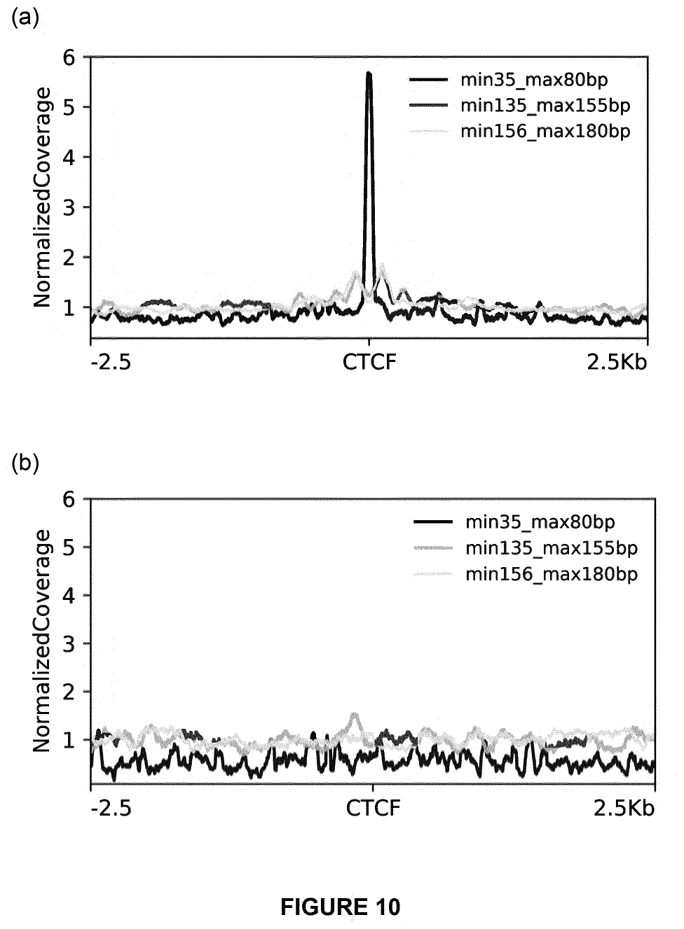

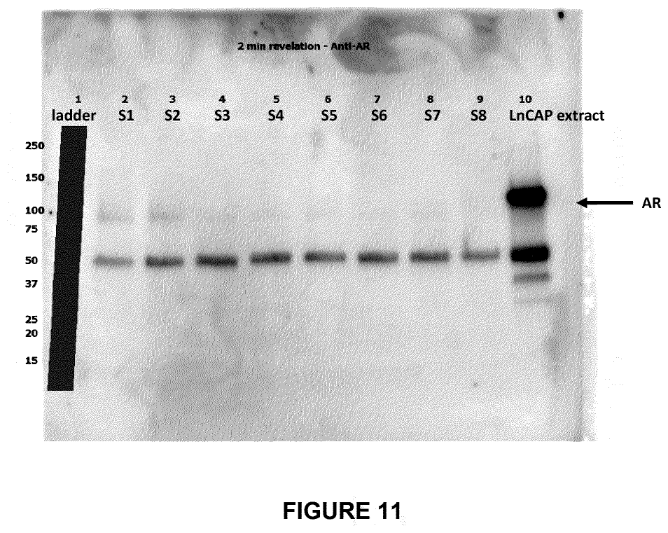

The invention finds particular use in analysing small DNA fragments bound by transcription factors, usually in the size range of 35-80 bp. Therefore, in one embodiment the extracted DNA that is sequenced relates to small DNA fragments, such as DNA fragments comprising less than about 100 bp, such as less than about 80 bp, in particular less than about 60 bp. It is noted that these DNA fragment sizes relate to the DNA fragments without/prior to adapter ligation. In one embodiment the extracted DNA that is sequenced comprises DNA fragments in the size range below 100 bp, such as 35-80 bp (without/prior to adapter ligation). In one embodiment the extracted DNA that is sequenced contains a plurality of DNA size ranges which are then compared, for example as shown in FIGS. 10 and 11.

In preferred embodiments the sample is a body fluid sample. In a further embodiment, the body fluid sample is a blood, serum or plasma sample.

In preferred embodiments, the binding agent used is an antibody directed to bind to a particular transcription factor. Thus, in one embodiment, the binding agent which binds to the transcription factor is an antibody or a fragment (i.e. a binding fragment) thereof.

In preferred embodiments, the antibody is immobilized on a solid phase to facilitate isolation of antibody bound transcription factor-DNA complexes or chromatin fragments.

The presence of both a transcription factor together with an associated DNA fragment of a sequence that is known to be consistent with the transcription factor in vivo, in a circulating chromatin fragment is further confirmation of the identity of both the transcription factor and the DNA fragment. This combination of a transcription factor together with the sequence of the associated DNA fragment is a powerful biomarker combination for the diagnosis or assessment of a wide variety of disease conditions. Further, many transcription factors that are present in healthy subjects are bound to a different set of TFBS in different tissues so identifying the TFBS locations bound by a transcription factor through the associated DNA present, identifies the tissue of origin of the chromatin fragments. Moreover, the same applies to disease conditions. Thus, the presence of a disease condition may be identified from the set of TFBS bound to a commonly expressed transcription factor (even though the transcription factor itself is expressed in many or all tissues). For example, the commonly expressed transcription factor CTCF binds to more than a thousand specific genomic locations in immortalized cancer cells but not in other non-cancer cells (Wang et al, 2012, Liu et al, 2017). Therefore, identifying the presence of a circulating CTCF-DNA complex wherein the associated DNA fragment is sequenced and observed to be of a sequence consistent with one of the cancer specific TFBS locations for CTCF is indicative of a cancer disease in the subject from whom the sample was obtained. Therefore, in a highly preferred embodiment of the invention there is provided a method for detecting a disease state in a subject by means of detecting a cell free chromatin fragment comprising a transcription factor and a DNA fragment which together form a combined biomarker that identifies that the transcription factor occupied TFBS location in the genome that is consistent with a disease condition or a particular tissue, in a body fluid sample obtained from a human or animal subject which comprises the steps of:
(i) contacting the sample with a binding agent which binds to a transcription factor;
(ii) isolating the bound transcription factor;
(iii) extracting the DNA associated with the transcription factor;
(iv) amplifying the extracted DNA;
(v) sequencing the amplified extracted DNA; and
(vi) using the sequence of associated DNA fragments as an indicator of the tissue of origin of the chromatin fragments or of a disease state in the subject.

Determination of the disease state of a subject may include, for example, the detection, diagnosis, treatment selection, monitoring or prognosis of or for a disease.

In one embodiment, the method comprises using the transcription factor and sequence of the associated DNA as a combined biomarker for indicating the presence of the disease in the subject. The term “biomarker” means a distinctive biological or biologically derived indicator of a process, event, or condition. Biomarkers can be used in methods of diagnosis, e.g. clinical screening, and prognosis assessment and in monitoring the results of therapy, identifying subjects most likely to respond to a particular therapeutic treatment, drug screening and development. Such biomarkers include, for example, the presence (e.g. sequence), level, concentration or amount of DNA associated with a transcription factor. References herein to a “combined biomarker” refer to a biomarker which involves more than one biological, or biologically derived, indicator, e.g. a transcription factor and associated DNA, in particular the level, concentration or amount of transcription factor associated with a particular sequence, or sequences, of DNA.

Tissue specificity is important because most transcription factors do not have perfect (single cell type) specificity of expression. The tissue specificity of an immunoassay for circulating chromatin fragments containing a transcription factor is limited both by the analytical specificity of the antibody used and by the tissue specificity of the transcription factor used, or the panel of transcription factors used. Therefore, the tissue specificity can be improved by combining the particular transcription factor moiety with the sequence of the cfDNA fragment to which it is bound.

The reason for this is that transcription factors bind to different DNA sequences in the genome in different cells. Gene expression is regulated by specific binding of transcription factors to short TFBS DNA sequences, also referred to as response elements or binding motifs. The TFBS is typically, but not necessarily, located in a gene promoter region near to the transcription start site of the regulated gene. Transcription factors bind to the TFBS in a sequence specific manner through a DNA Binding Domain (DBD). Typically, a TFBS sequence is 5-15 bp long within the promoter of its target gene and a transcription factor protein can usually bind to a set of similar DNA sequences with varying degrees of binding affinity. The length of DNA fragments associated with circulating chromatin fragments containing transcription factors will vary depending on whether the fragment also includes further DNA protected sequences bound by further transcription factors, cofactors, nucleosomes or other chromatin proteins. Many such chromatin fragments are reported to occur in the 35-80 bp range (Snyder et al, 2016). Furthermore, we note that this agrees with the size range of chromatin fragments produced by nuclease digestion of chromatin extracted from the cells of cancer patients and that this small approximately 35-80 bp fragment range comprises a greater proportion of total chromatin fragments than nucleosome bound fragments (Corces et al, 2018). We conclude that these associated DNA fragments are longer than typical DNA response elements and therefore include flanking DNA sequences. However, the DNA fragment size associated with a nucleosome typically exceeds 100 bp DNA. We therefore conclude that the 35-80 bp DNA fragment range does not include intact nucleosomal DNA fragments.

The response element, or TFBS sequences, of a transcription factor may occur repeatedly in many locations within the genome, and occurs in thousands of locations for some transcription factors. There is, therefore, the potential for the same transcription factor to be bound in a great many locations within the chromatin of a cell. This means that the death of a single cell may, in principle, give rise to a large number of circulating chromatin fragments containing the same transcription factor.

Moreover, transcription factors tend not to act alone but in concert with other transcription factors or co-factors or other moieties that are required for the regulation of a particular gene. Thus, a transcription factor may bind to a response element in the promoters of a large number of different genes, each in concert with different transcription factors. Thus, the DNA flanking sequence surrounding the same TFBS sequence or response element for the same transcription factor, varies in the promoters of different genes because it includes the binding motifs for different combinations of transcription factors. This applies to all or most transcription factors.

In addition, the binding sequence of the response element itself may be degenerate so that the transcription factor may bind to a variety of different motif sequences. For example, the transcription factor TTF-1 is expressed in a tissue specific manner in healthy lung and healthy thyroid tissue. In lung, two protein TTF-1 factors bind to the promoter region of the lung-specific Surfactant Protein B (SPB) gene. The DNA binding sequence, or binding motif, of TTF-1 in the promoter of SPB is GCNCTNNAG (SEQ ID NO: 1) (where A, C, G and T denote the DNA bases adenine, cytosine, guanine and thymine respectively and N denotes any of these bases). The wider consensus promoter DNA sequence surrounding the TTF-1 binding is (−118)GATCAAGCACCTGGAGGGCTCTTCAGAGCAAAGACAAACACTGAGGTCGCTGC CA(−64) (SEQ ID NO: 2), where (−64) denotes the distance in bp from the SPB transcription start site. In the SPB promoter in lung tissue, TTF-1 binds in concert with the transcription factor Hepatocyte Nuclear Factor 3 (HNF3) as shown in FIG. 1 (Matys et al, 2006 and Bohinski et al, 1994).

In the thyroid, TTF-1 regulates a number of genes including thyroglobulin, thyroid stimulating hormone receptor and thyroperoxidase. The consensus binding sequence for TTF-1 in the promoter region of thyroglobulin gene is different to than that in lung and is reported as TGGCCACACGAGTGCCCTCA (SEQ ID NO: 3). In the promoter of the thyroglobulin gene, TTF-1 binds cooperatively with TTF-2, PAX8 and Runx2 transcription factors and the wider sequence including 50 bp flanking sequences at the 5′ and 3′ ends is CCCACCCCGTTCTGTTCCCCCACAGTTTAGACAAGATCCTCATGCTCCACTGGCCACA CGAGTGCCCTCAGGAGGAGTAGACACAGGTGGAGGGAGCTCCTTTTGACCAGCAGA GAAAAC (SEQ ID NO: 4). Similarly, TTF-1 also binds to the promoter regions of the thyroid stimulating hormone receptor and thyroperoxidase genes in concert with different cooperating transcription factors in each case. Thus, not only does the sequence of DNA surrounding the TTF-1 binding site in the promoter sequence of genes regulated in thyroid or lung tissue differ, but the cofactors associated with TTF-1, and hence the surrounding DNA sequence, also differs for binding to different genes in the same tissue as shown in FIG. 1 (Matys et al, 2006 and Maenhaut et al, 2015). This demonstrates that the combination of a circulating chromatin fragment containing TTF-1, together with the knowledge of the DNA sequence associated with the chromatin fragment, is sufficient to identify the origin of the chromatin fragment as lung or thyroid.

There are thought to be approximately 1000-3000 human transcription factors each of which binds specific locations in the genome resulting in dynamic transcriptional changes that drive a vast array of cellular processes. We have illustrated the principle of the invention with respect to TTF-1 as one example. However, any transcription factor may in principle be used in methods of the invention. Even, transcription factors that are ubiquitously expressed in many cell types and bind discreet DNA sequences, for example Hox protein transcription factors, bind cooperatively with cofactors to uniquely bind to different sequences to regulate different genes in different tissues (Merabet and Mann, 2016, Mann et al, 2009). This means that all or most transcription factors together with their TFBS sequences (optionally including flanking sequences) may be used as combination biomarkers for the methods of the invention. For example, the estrogen receptor-α (ERα) transcription factor binds to more than a thousand binding sites or estrogen response elements (ERE) in the human genome in concert with combinations of at least 60 other transcription factors at different genomic locations (Lin et al, 2007). Similarly, the androgen receptor (AR) binds the androgen response element (ARE) associated with thousands of genes in concert with other cooperating transcription factors at thousands of distinct different sequence loci. Thus, methods of the invention may identify the tissue of origin of a chromatin fragment containing ERα or AR through the sequence of associated DNA even though these transcription factors are expressed in multiple tissues.

Moreover, the genome wide binding of transcription factors to DNA loci is reprogrammed in cancer and the transcription factors expressed and the TFBSs they bind to in cancer cells differ from those bound in healthy cells of the same tissue, so the identification of a chromatin fragment containing a transcription factor in the circulation in combination with the sequence data of the associated DNA fragment, enables both the identification of a subject with a cancer as well as the identification of the cancer type, for example as a prostate cancer or a lung cancer etc. (Pomerantz et al, 2015). This is enabled because chromatin is remodeled during tumorigenesis and this remodeling involves upregulation of tumor associated proteins through remodeled transcription factor binding patterns in the cancer cell. Because of this, the expression of many transcription factors is upregulated in cancer cells. This is a broad phenomenon, but can be exemplified by a few, non-limiting examples. For example, the well known cancer associated transcription factors c-Myc and p53 are upregulated in most cancers. The binding site sequences bound by AR are greatly altered in prostate cancer (Pomerantz et al 2015). Similarly, the epithelial to mesenchymal transition (EMT) in cancer cells, which is associated with metastasis and resistance to therapy, involves the upregulation of the Jun/Fos family of transcription factors, including Fosll, Fosb, Fos, and Junb. The ETS (E26 transformation-specific) family of transcription factors as well as the Runxl, Tead and Nfkb transcription factors, have also been found to be highly enriched in the open chromatin of tumor cells. In addition, p63, Klf, Grhl, and Cepba are reported to be upregulated in tumor cells, and their binding sites are enriched in the open chromatin regions. Klf5 and p63 transcription factors are associated with carcinomas and act as drivers in lung and head and neck carcinomas. Further transcription factors associated with EMT include bHLH, Runx, Nfat, Tbx1, Tcf711 and Smad2 (Latil et al, 2017)

The regulation of transcription of eukaryotic genes involves a multiplicity of regulatory proteins bound to a multiplicity of regulatory DNA sequences, located both near to the transcription start site (TSS) of the gene and distal to the TSS in the genome in a transcription complex, for example as illustrated in FIG. 2. The distal regulatory sequences in the DNA may be located a few hundred to more than a million bases from the TSS or may be more distant. The transcription complex typically involves a loop of DNA, which may involve a DNA bending protein, wherein the more distal regulatory sequences, as well as the regulatory proteins bound to them, are brought into contact with the proteins that are bound to the regulatory sequences nearer to the TSS, for example as also illustrated in FIG. 2. The TATA box is so named because it contains a sequence of repetitive Thymine/Adenine nucleotides that bind to general transcription factors required for transcription. Further gene specific transcription factors are also required for the expression of the particular gene (for example the transcription factors required to express the surfactant protein B, thyroglobulin, thyroperoxidase and TSH receptor genes as shown in FIG. 1). In addition, a multiplicity of other proteins are necessary including, for example without limitation, co-factors, mediators, activators, co-activators, repressors, co-repressors, chromatin remodeling proteins, DNA bending proteins, insulators, RNA polymerase moieties, elongation factors, chromatin remodeling factors, STAT moieties or cytokine factors or cytokine related factors bound to a STAT moiety, Upstream Binding Factor (UBF) or any other moieties associated with such a gene regulation or transcription complex. Such complexes may also include lengths of nucleosome protected DNA. Transcription complexes can be stable to facilitate high volume transcription. Therefore, circulating chromatin fragments of healthy and/or disease origin may include large protein/DNA complexes that comprise multiple proteins which may be resistant to nuclease activity. Some large transcription complexes involving near and distal regulatory sequences, as illustrated in FIG. 2, are termed super-enhancers. Super-enhancers are large clusters with high levels of transcription factor binding and are central to driving the expression of genes involved in controlling cell identity. Super-enhancers are also central to stimulating transcription of oncogenes in cancer. Cancer cells acquire super-enhancers and cancerous phenotypes rely on abnormal transcription driven by super-enhancers. Therefore, detection of the presence of chromatin fragments including all or parts of super-enhancer complexes and/or combinations of cfDNA fragment sequences that correspond to the near and distal regulatory sequences of super-enhancers by the methods described herein provides a method of identifying the cellular origin of chromatin fragments including cancer cells of origin. We also reason that by their nature, super-enhancer complexes are likely to comprise stably bound, rather than transiently bound, transcription factors.

The loop of DNA in such a chromatin fragment derived from a transcription complex may in principle either be intact, or may be digested at one or more locations, resulting in either (i) two circulating chromatin fragments corresponding to the near and distal regulatory sequences; or (ii) a large chromatin fragment that contains two fragments of DNA. Therefore, cfDNA may include small DNA fragments that correspond to both the near and distal regulatory sequences of a gene.

According to a further aspect of the invention, there is provided a method of detecting a disease in a human or animal subject which comprises the steps of:
(i) contacting a body fluid sample obtained from the human or animal subject with a binding agent which binds to a transcription factor;
(ii) determining the sequence of one or more DNA fragments associated with the transcription factor; and
(iii) using the presence of the transcription factor and the sequence of the associated DNA as a combined biomarker for determining the presence and/or nature of the disease in the subject.

It will be understood that any non-histone chromatin protein that binds to DNA and whose cfDNA binding pattern is different in healthy and diseased subjects will be suitable for use in methods of the invention, including transcription factors as well as other non-histone chromatin proteins including chromatin modifying proteins, genetic and epigenetic reading, writing and deleting proteins, proteins involved in RNA transcription (for example RNA polymerase molecules) and architectural or structural chromatin proteins (for example DNA bending proteins).

Therefore, according to a further aspect of the invention, there is provided a method of detecting a disease in a human or animal subject which comprises the steps of:
(i) contacting a body fluid sample obtained from the human or animal subject with a binding agent which binds to a non-histone chromatin protein;
(ii) determining the sequence of one or more DNA fragments associated with the non-histone chromatin protein; and
(iii) using the presence of the non-histone chromatin protein and the sequence of the associated DNA as a combined biomarker for determining the presence and/or nature of the disease in the subject.

In a preferred embodiment the non-histone chromatin protein is RNA polymerase, in particular RNA polymerase II. RNA polymerase II is a DNA binding enzyme which is responsible for transcribing the DNA sequence of a gene to produce an RNA copy. The RNA copy may be a messenger RNA (mRNA) molecule leading to corresponding protein production by ribosomes, or may be a non-coding RNA (ncRNA) molecule that is not translated into a protein. The presence of RNA polymerase II in a circulating chromatin fragment therefore indicates that the fragment derives from a gene which was active in the cells from which it originated. A library of DNA fragment sequences derived from chromatin fragments associated with RNA polymerase II therefore provides a library of active dynamic genes present in the sample. In a healthy person, this library corresponds mostly to the active genes present in hematopoietic tissues. In a diseased person the library additionally includes genes active in the tissue(s) affected by the disease. This may be any tissue affected by disease. For example, genes active in liver or kidney cells may be represented in the RNA polymerase II library produced from samples taken from patients with liver or kidney disease, where such genes are not present in the library of a healthy person. Similarly, genes upregulated in cancer may be represented in the RNA polymerase II library produced from samples taken from patients with a cancer disease, where such genes are not present in the library of a healthy person. Use of RNA polymerase II in this aspect of the invention allows for the identification of the active dynamic genes represented in the sample. This allows for the detection of cancer diseases as well as the determination of the tissue(s) affected by the cancer.

Therefore, according to a further aspect of the invention, there is provided a method of detecting a disease in a human or animal subject which comprises the steps of:
(i) contacting a body fluid sample obtained from the human or animal subject with a binding agent which binds to RNA polymerase;
(ii) determining the sequence of one or more DNA fragments associated with RNA polymerase; and
(iii) using the sequence of the RNA polymerase associated DNA fragment as a biomarker for determining the presence and/or nature of the disease in the subject.

In one embodiment, the disease is selected from cancer, an autoimmune disease or inflammatory disease. In a further embodiment, the disease is cancer. In a further embodiment, the autoimmune disease is selected from: Systemic Lupus Erythematosus (SLE) and rheumatoid arthritis. In a further embodiment, the inflammatory disease is selected from: Crohn's disease, colitis, endometriosis and Chronic Obstructive Pulmonary Disorder (COPD).

In preferred embodiments, the disease is cancer. In a further embodiment, the cancer is selected from: breast cancer, bladder cancer, colorectal cancer, skin cancer (such as melanoma), ovarian cancer, prostate cancer, lung cancer, pancreatic cancer, bowel cancer, liver cancer, endometrial cancer, lymphoma, oral cancer, head and neck cancer, leukemia and osteosarcoma.

In further embodiments the disease is a fetal disease or condition. It is well known in the art that chromatin fragments of fetal origin, for example containing Y-chromosome DNA sequences originating from a (XY) male fetus, circulate in the blood of pregnant animal and human (XX) mothers. The cfDNA circulating in pregnant subjects has been reported to comprise both cfDNA fragments of the length expected of nucleosome protected DNA fragments (approximately 160 bp) as well as shorter cfDNA fragments in the range 50 bp upwards. Moreover, it has been reported that maternal cfDNA fragments of less than 140 bp in length are enriched for cfDNA of fetal origin (Hu et al; 2019). Thus, methods of the present invention are applicable not only to disease states of the subject from whom the sample was taken, but also to the prenatal investigation or testing of fetal conditions in maternal blood samples.

Therefore, according to a further aspect of the invention, there is provided a method of detecting a disease in a human or animal fetus which comprises the steps of:
(i) obtaining a body fluid sample from a pregnant human or animal subject;
(ii) contacting the body fluid sample with a binding agent which binds to a transcription factor;
(iii) detecting, measuring or sequencing the DNA associated with the transcription factor; and
(iv) using the presence, sequence or amount of DNA as an indicator of the presence of a disease in the fetus.

According to a further aspect of the invention, there is provided a method of detecting the tissue affected by a disease in a human or animal subject which comprises the steps of:
(i) contacting a body fluid sample obtained from the human or animal subject with a binding agent which binds to a transcription factor;
(ii) determining the DNA base sequence of the DNA associated with the transcription factor or chromatin fragment; and
(iii) using the combined transcription factor/DNA sequence biomarker as an indicator of the tissue affected by the disease in the subject.

In a preferred embodiment, the disease is cancer. In another embodiment, the tissue affected by the disease is the organ of origin, such as the organ of origin of a cancer.

In another aspect of the invention, there is provided a method of detecting a disease in a human or animal subject which comprises the steps of:
(i) contacting a body fluid sample obtained from the human or animal subject with a binding agent which binds to a transcription factor;
(ii) isolating the DNA associated with the transcription factor;
(iii) amplifying the isolated DNA by a PCR method;
(iv) determining the sequence of the amplified DNA; and
(v) using the presence of the transcription factor and the sequence of the associated DNA as a combined biomarker for determining the presence and/or the nature of a disease in the subject.

It will also be clear to those skilled in the art that a multiplicity of sequences may be obtained corresponding to various gene loci bound by the particular transcription factor and the data regarding the various sequences may be integrated to determine the nature of the disease and/or the tissue affected by the disease.

In another aspect of the invention, there is provided a method of detecting a disease in a human or animal subject which comprises the steps of:
(i) contacting a body fluid sample obtained from the human or animal subject with a binding agent which binds to a transcription factor;
(ii) isolating the DNA associated with the transcription factor;
(iii) amplifying the isolated DNA by a PCR method, e.g. using sequence specific primers;
(iv) detecting the amplified DNA; and
(v) using the presence, amount and/or sequence of amplified DNA as an indicator of the presence and/or the nature of a disease in the subject.

In one embodiment, amplification of the isolated transcription factor bound DNA fragment is performed following ligation of an adapter oligonucleotide to the DNA fragment. Therefore, in one embodiment of the invention there is provided a method of detecting a disease in a human or animal subject which comprises the steps of:
(i) contacting a body fluid sample obtained from the human or animal subject with a binding agent which binds to a transcription factor;
(ii) isolating the DNA associated with the transcription factor;
(iii) ligating an adapter oligonucleotide to the isolated DNA;
(iv) amplifying the DNA; and
(v) using the presence, amount and/or sequence of the DNA fragment as an indicator of the presence and/or the nature of a disease in the subject.

In another aspect of the invention, there is provided a method of detecting a disease in a human or animal subject which comprises the steps of:
(i) contacting a body fluid sample obtained from the human or animal subject with a binding agent which binds to a transcription factor;
(ii) isolating the DNA associated with the transcription factor;
(iii) amplifying the isolated DNA using sequence specific primer oligonucleotides;
(iv) detecting the amplified DNA; and
(v) using the presence, amount and/or sequence of amplified DNA as an indicator of the presence and/or the nature of a disease in the subject.

This aspect utilizes the tissue specificity of the combined transcription factor/DNA sequence biomarker of the invention whilst obviating DNA fragment adapter library preparation and next generation DNA sequencing by PCR amplification of selected DNA fragments including the TFBS sequence(s) and/or flanking sequence(s) of interest for biomarker purposes. The method is rapid, low cost, easily automated for high throughput and may be performed in any PCR laboratory.

The DNA sequences isolated in steps (i) or (ii) may be amplified by any method known in the art. In some embodiments isolated DNA is amplified using a PCR method employing adapters which are ligated to the DNA fragments. In other embodiments PCR primers are used for DNA amplification. Primers may be designed to amplify all DNA sequences isolated in steps (i) or (ii), or may be designed to amplify specific DNA sequences associated with the sequence of a response element of a transcription factor, optionally also including flanking regions.

In another aspect of the invention, there is provided a method of detecting a disease in a human or animal subject which comprises the steps of:
(i) contacting a body fluid sample obtained from the human or animal subject with a binding agent which binds to a transcription factor;
(ii) isolating (and optionally amplifying) the DNA associated with the transcription factor;
(iii) detecting the DNA by a hybridization method; and
(iv) using the presence, amount and/or sequence of DNA hybridized as an indicator of the presence and/or the nature of a disease in the subject.

This aspect utilizes the tissue specificity of the combined transcription factor/DNA sequence biomarker of the invention whilst obviating expensive next generation DNA sequencing by selective DNA hybridization of DNA fragments including the TFBS sequence(s) and/or flanking sequence(s). The method is low cost and may be performed in any PCR laboratory.

In preferred embodiments, the isolated DNA is amplified prior to hybridization. In preferred embodiments the hybridization method is a DNA microarray method (also known as a DNA chip method).

The method of the invention may also be used to measure the combined biomarker of the transcription factor and sequence-associated DNA.

### Selection of Transcription Factors

The regulation of gene transcription in eukaryotic organisms is highly complex and may involve bending and looping of DNA to bring together multiple regulatory DNA sequences bound by multiple regulatory proteins in a regulatory transcription complex as illustrated in FIG. 2. The term “transcription factor” as used herein therefore means a regulatory protein that binds directly or indirectly to a gene regulatory sequence in the genome to regulate the transcription of a gene including, without limitation, general transcription factors and specific transcription factors associated with the regulation of particular gene(s) as well as enhancer, co-enhancer, repressor, co-repressor, mediator, activator, co-activator, repressor, co-repressor, chromatin remodeling protein, DNA bending protein, insulator, RNA polymerase moiety, elongation factor, STAT moiety, cytokine factor or cytokine related factor bound to a STAT moiety, UBF or any other moieties associated with such a gene regulation or transcription complex. Similarly, the term “transcription factor binding site” (TFBS) as used herein means a DNA binding site of a regulatory protein associated with transcription regulation of a gene including without limitation distal or proximal enhancer and repressor sequences as shown in FIG. 2.

It is well known that transcription factor expression is altered in disease. Thus, a method of the invention may relate to a transcription factor whose expression is upregulated in disease, and/or inappropriately expressed in a disease tissue, for example a cancer tissue, when usually not highly expressed in said (healthy) tissue. Therefore the level of a transcription factor present in a body fluid sample may be used as a biomarker of disease.

It is also well known that the profile of TFBS occupancy by transcription factors is altered in different cell types and in disease (Wang et al, 2012). Therefore the profile of TFBS occupancy by a transcription factor present in a body fluid sample may be used as a biomarker of disease.

The chromatin fragments present in the circulation of healthy subjects are predominantly of hematopoietic origin. Thus, a method of the invention also relates to detecting the inappropriate presence of a chromatin fragment comprising a transcription factor together with associated DNA which is not normally expressed in haemopoietic tissues (but may be expressed in a non-hematopoietic tissue).

For example, many cancer diseases are derived from epithelial tissues. The epithelial GRHL2 transcription factor is expressed in many epithelial tissues as well as in many epithelial tissue derived cancer diseases, but is not expressed in hematopoietic tissues. The presence of GRHL2 in the circulation indicates the presence of an epithelial derived cancer, for example a colorectal, prostate, lung or breast cancer. Thus, methods of the invention may be used to detect the presence of cancer per se, as well as identifying the organ of origin of the cancer using lineage specific transcription factors and/or lineage specific combinations of transcription factors with associated DNA sequences. Any transcription factor may therefore be useful in methods of the invention. In preferred embodiments, the level of chromatin fragments including the transcription factor selected is elevated in a body fluid of diseased subjects (over levels found in other subjects), is partially or wholly tissue and/or disease specific, and/or has multiple response elements in the genome.

Therefore, in one embodiment, the transcription factor is disease specific (i.e. the level of circulating chromatin fragments including the transcription factor is upregulated in disease). In one embodiment, the transcription factor is tissue specific. In one embodiment, the transcription factor binds at more than one position in the genome, such as more than 5, more than 10, more than 100, more than 1000 or more than 10,000 positions in the genome. Some transcription factor binding positions are occupied in some tissue types but not in others. Some transcription factor binding positions are occupied in diseased cells but not in healthy cells of the same tissues.

Transcription factors may be classified by binding domain (e.g. see Vaquerizas et al, 2009 which is incorporated herein by reference). In one embodiment, the transcription factor comprises a DNA binding domain selected from: a homeodomain, a HLH, a bZip, a NHR, a Forkhead, a P53, a HMG, an ETS, aIPT/TIG, a POU, a MAD, a SAND, a IRF, a TDP, a DM, a Heat shock, a STAT, a CP2, a RFX, an AP2 or a zinc finger (e.g. zinc finger C2H2 or zinc finger GATA) binding domain. In one embodiment, the transcription factor comprises a non-zinc finger DNA binding domain.

Suitable transcription factors may be determined experimentally, for example using classical Nuclease Accessible Site mapping methods to identify transcription factors of interest in the tissue(s) of interest. In a typical experiment, chromatin is extracted from the cells of interest (for example a cancer cell, a healthy cell of the same tissue, and a haemopoietic cell) and digested using a suitable nuclease. The chromatin fragments produced by digestion are exposed to an antibody that binds to a transcription factor and the antibody bound DNA fragments are isolated and sequenced to identify the TFBS sequence(s) (optionally including flanking sequences) bound by the transcription factor. The results can be used to select transcription factors for use in the invention. For example, transcription factors and transcription factor/TFBS (optionally including flanking sequences) combinations that are elevated in diseased cells but low or absent in hematopoietic cells are useful in methods of the invention. Classical nuclease accessibility methods have recently been improved upon and the art now includes methods, for example, CUT&RUN and other methods, which are simpler to perform and provide improved results (Skene and Henikoff, 2017). Any such methods will be suitable for use in the identification of suitable transcription factors for use in the present invention.

Many such experiments, and similar experiments, have been performed and suitable transcription factors are thus available in the art. There are many publications on transcription factors and cancer in the literature that list transcription factors useful in methods of the invention. For example Lambert et al, 2018 lists 294 known oncogenic transcription factors and regulators. Gurel et al, 2010 describes the transcription factor NKX3.1 as a marker for prostate cancer. Damell, 2002 lists a number of oncogenic transcription factors including STAT3, 5, STAT-STAT, GR, IRF, TCF/LEF, β-catenin, NF-κB, NOTCH (NICD), GLI, c-JUN, bZip proteins (including c-JUN, JUNB, JUND, c-FOS, FRA, the ATFs and the CREB-CREM family), the cEBP family, ETS proteins and the MAD-box family. Vaquerizas et al, 2009 describe a number of tissue specific transcription factors useful in methods of the invention. Ulz et al, 2019 describes transcription factors such as the epithelial transcription factor GRHL2 which is present in many cancer types but not in hematological tissues as well as AR (Androgen Receptor), NKX3-1 and HOXB13. Corces et al, 2018 describe a number of cancer specific and tissue specific transcription factors including NR5A1, TP63, GRHL1, FOXA1, GATA3, NFIC, CDX2, RFX2, ASCL1, PAX2, HNF1A, NKX2.A, PHOX2B, DRGX, HOXB13, AR, MITF, HNF4 and POU5F1. Using ChIP-Seq, Wang et al, 2012 identified 77,811 distinct binding sites for the transcription factor CTCF across 19 different cell types, including 7 immortal cancer cell lines and 12 normal cell types. Of these 77,811 CTCF TFBS, 1236 sites were found to be differentially occupied in cancer cells. Occupation of 195 sites was found to occur in normal cell types but not in cancer cells. Occupation of 1041 sites was found to occur in cancer cells but not in normal cell types (Liu et al, 2017). The finding of CTCF associated cfDNA fragments corresponding to cancer specific TFBS in a body fluid by ChIP-Seq is indicative of the presence of a cancer disease in the subject investigated and can be used as a biomarker in this manner. Said references are herein incorporated by reference.

Suitable transcription factors for use with the method of the invention may also be selected using various transcription factor, cancer and genomic databases, for example the ENSEMBL database which provides an annotated genome sequence for a number of species including humans, the Encyclopedia of DNA Elements or (ENCODE) database (https://www.encodeproject.org), the Transcription Factor (TRANSFAC) database (Matys et al, 2006), The Gene Transcription Regulation Database (GTRD) Version 18.01 (http://gtrd.biouml.org), the Human Transcription Factors database Version 1.01 (http://humantfs.ccbr.utoronto.ca), the NIH Genomics Data Commons database (https://gdc.cancer.gov), The Cancer Genome Atlas (TCGA) (https://www.cancer.gov/about-nci/organization/ccg/research/structural-genomics/tcga), the UCSC Xena Browser the (https://atacseq.xenahubs.net) and Human Protein Atlas database (https://www.proteinatlas.org) which provides data on the healthy tissues in which a transcription factor is expressed as well as its expression in cancer diseases, as well as other databases.

The use of these databases for the characterization of transcription factors and associated TFBS sequences and flanking sequences for use in methods of the invention, can be illustrated with reference to a few of these databases as an example. The TRANSFAC database provides data on many thousands of human and other eukaryotic transcription factors. Details provided for each transcription factor include the number of TFBSs it binds to in the genome, lists of genes whose transcription it regulates, the sequence and genomic position of TFBSs associated with each regulated gene, details of other transcription factors that operate with it in a cooperative manner to regulate transcription, consensus TFBS DNA sequences, DBD details and cancer association. The use of this data in the context of the present invention is exemplified below for the transcription factors CDX2 and c-JUN for illustrative purposes. The TRANSFAC database lists 48 human CDX2 TFBSs which regulate 26 specified genes. The CDX2 TFBS sequences are provided as well as their genomic location and the genes regulated by each. The flanking sequences for each CDX2 TFBS can be determined by reference to the ENSEMBL human genome database for the sequence at each genomic location. Consensus CDX2 TFBS sequences are also provided. Similarly, The TRANSFAC database lists 265 human c-JUN TFBSs which regulate 166 specified genes. The c-JUN TFBS sequences are provided as well as their genomic location and the genes regulated by each. The flanking sequences for each c-JUN TFBS can be determined by reference to the ENSEMBL human genome database for the sequence at each genomic location. Consensus c-JUN TFBS sequences are also provided.

Therefore, a transcription factor and/or TFBS may be selected experimentally or from the literature and/or from databases, such as The Human Protein Atlas database, as useful in methods of the invention. The transcription factor may be characterized in terms of (i) the healthy and diseased tissues in which it is expressed, (ii) the genes regulated in those cells or tissues, (iii) the TFBS sequences (optionally including flanking sequences) to which it binds in those tissues and (iv) other factors with which it cooperates by co-binding on a TFBS for transcriptional regulation. This characterization may be used to identify the healthy or diseased tissue or cells of origin of chromatin fragments and/or transcription factor associated cfDNA fragments in a body fluid sample, by the methods described herein.

Similarly, experimental data relating to chromatin fragments and/or cfDNA sequences in body fluid samples may be interpreted using these databases to identify all or part of a TFBS sequence, optionally including flanking sequences, included in a cfDNA fragment. This data may then be used to identify the tissue or cells of origin of the cfDNA fragment.

There are three main groups of transcription factors which are currently recognized as being particularly important in cancer. The first group is the nuclear hormone receptor group which includes the estrogen receptor, the androgen receptor, the progesterone receptor, the glucocorticoid receptor, the thyroid receptor and the retinoic acid receptor. The nuclear hormone receptor group of transcription factors are cell surface receptors which can be regarded as inactive or latent transcription factors that may be activated by ligand binding. For example, the estrogen receptor is activated by binding to estrogen. Ligand binding results in migration of the nuclear hormone receptor to the nucleus where it binds to the target DNA sequence (for example, the estrogen receptor binds to the estrogen response element) and up or down regulates genes associated with the DNA target sequence (for example, estrogen regulated genes).

The second group of transcription factors that are known to be important in the initiation and development of cancer are the signal transducers and activators of transcription (STATs). These are latent cytoplasmic transcription factors that may be activated by a large variety of molecular triggers in the cytoplasm and/or at the cell surface. STAT activation typically involves a cascade of biochemical events in the cytoplasm such as kinase reactions, proteolysis reactions and protein-protein interactions that result in entry to the nucleus of a protein, or protein complex, that modulates transcription of target genes. Often the biochemical cascade leading to activation of transcription, is triggered by receptor binding of a ligand at the cell surface including for example, binding of a cytokine moiety by a cytokine receptor, or binding of a growth factor such as epidermal growth factor or platelet derived growth factor by a growth factor receptor, or by binding of a peptide or protein to a G protein-coupled receptor.

The third group of transcription factors important in cancer are resident nuclear proteins whose transcriptional effects are typically activated by a cascade of biochemical events involving serine kinase reactions. There are hundreds of serine kinase moieties and hundreds of nuclear proteins that are targets for serine kinases.

It will be clear to those skilled in the art that cell free chromatin fragments comprising (i.e. including or containing) any transcription factor involved in the initiation, development or maintenance of cancer, such as transcription factors in the three groups described above, will be useful in the methods of the present invention. Some transcription factors, or transcription factor families, with known roles in cancer, or known to be elevated in cancer diseases include for example, without limitation, STAT, particularly STAT3, STAT5 and STAT-STAT dimer moieties, NF-κB, β-catenin, γ-catenin, Notch and notch intracellular domain (NICD), GLI, c-JUN, JUNB, JUND, c-FOS, FRA, ATF, CREB-CREM, cEBP, ETS, MYC, N-MYC, MAX, E2F, interferon regulatory factor (IRF), T-cell factors (TCF), lymphocyte enhancer factors (LEF), EN2, GATA3, CDX2, PAX8, WT1, NKX3.1, P63 (TP63) or P40 and helix-loop-helix proteins (Darnell, 2002). All such transcription factors will be useful in methods of the invention.

It has been found that many transcription factors are lineage specific and associated with specific tissues and therefore could be considered tissue specific transcription factors, i.e. a transcription factor that is always or commonly expressed in certain tissues or cancers whilst being rarely or never expressed in other tissues or cancers. Methods of the invention may be used with tissue specific transcription factors where the combined detection of the associated DNA provides enhanced specificity and/or sensitivity.

Thyroid transcription factor 1 (TTF-1) is selectively expressed during embryogenesis in the thyroid, the diencephalon, and in respiratory epithelium. TTF-1 is expressed in tissue samples taken from neuroendocrine and non-neuroendocrine lung carcinomas but its frequency of expression varies markedly among different histologic subtypes. Therefore, methods of the invention may also be used to identify cancer types and subtypes through the measurement of a chromatin fragment containing a transcription factor and its associated DNA sequence.

PAX8 is a transcription factor involved in the embryogenesis of the thyroid gland, kidney, and mullerian system. PAX8 shows a high level of expression in tissue samples taken from nonmucinous ovarian carcinomas, serous, endometrioid, clear cell, and transitional cell carcinomas. PAX8 is also expressed in endometrioid adenocarcinomas, uterine serous carcinomas, endometrial clear cell carcinomas as well as in ductal and lobular breast carcinoma tissues.

CDX2 is a lineage specific transcription factor with a key role in controlling the proliferation and differentiation of intestinal epithelial cells and is expressed in almost all colorectal adenocarcinoma tissue samples.

NKX3.1 is required for normal prostate development and is a known marker expressed in almost all prostate cancers.

GATA3 is active in transcription as early as the fourth week of human gestation. GATA3 is highly expressed in tissue samples taken from breast carcinomas, particularly estrogen receptor positive breast cancer tissue samples, and urothelial carcinomas and transitional cell carcinomas.

WT1 plays an important role in embryo development. WT1 is a good marker of ovarian cancer tissue and is expressed by a very limited range of healthy adult tissues.

EN2 has a role in embryological development and is expressed in a range of cancers but in very few adult healthy tissues. The presence of EN2 in the urine has been used as the basis for a urine test for the detection of prostate cancer.

Other transcription factors may be useful in the methods of the invention. For example, UBF is a transcription factor that binds to the ribosomal RNA gene promoter and activates transcription mediated by RNA polymerase I. UBF expression is known to be elevated in the tissue of some cancers. Many other such examples undoubtedly exist and are suitable transcription factors for use with methods of the present invention. Moreover, RNA polymerase I and RNA polymerase Ill are also elevated in cancers. These moieties are responsible for the transcription of tRNA and ribosomal RNA genes to provide the cellular machinery required for elevated and rapid protein production, growth and cellular replication characteristic of cancer cells and tissue. In further embodiments of the invention a method is provided for the detection or measurement of cell free chromatin fragments comprising UBF, RNA polymerase I or RNA polymerase III.

In alternative embodiments, the transcription factor is not a tissue-specific transcription factor. Methods of the invention are also able to detect transcription factors that are commonly expressed, i.e. a transcription factor which is expressed in more than 5, more than 10, more than 15, more than 20 or more than 30 tissue types. By combining detection with the associated DNA sequence (i.e. a combined biomarker), the methods of the invention may detect a commonly expressed transcription factor to provide a clinically useful result. Nuclear hormone receptor transcription factors are examples. As discussed above CTCF is also an example which was investigated further herein.

Transcription factors bind to their DNA target sequence in a highly cooperative fashion with many other factors including other transcription factors, cofactors, co-activators, co-repressors, RNA polymerase moieties, elongation factors, chromatin remodeling factors, mediators, STAT moieties, UBF and others. This means that the circulating transcription factors detected by the present invention may include other moieties as part of a larger gene regulation complex including any or all of a nucleosome with associated DNA, a nuclear hormone receptor, a steroid or other hormone bound to a nuclear hormone receptor, other transcription factors, cofactors, co-activators, co-repressors, RNA polymerase moieties, elongation factors, chromatin remodeling factors, mediators, STAT moieties or cytokine factors or cytokine related factors bound to a STAT moiety, upstream binding factor (UBF) or any other moieties associated with such a gene regulation or transcription complex that occurs in a cell free chromatin fragment.

Cell free chromatin fragments containing a transcription factor moiety may, or may not, also include the presence of an intact nucleosome or any histone proteins in the complex. All such cell free chromatin complexes will be useful in, and are included in, the present invention.

In a preferred embodiment, the transcription factor is selected from: STAT, NF-κB, β-catenin, γ-catenin, Notch, notch intracellular domain (NICD), GLI, c-JUN, JUNB, JUND, c-FOS, FRA, ATF, CREB-CREM, cEBP, ETS, MYC, MAX, E2F, interferon regulatory factor (IRF), T-cell factor (TCF), lymphocyte enhancer factor (LEF), and helix-loop-helix proteins, HOX protein, EN2, GATA3, CDX2, TTF-1, PAX8, WT1, NKX3.1, P63 (or TP63), P40 or CTCF. In a further embodiment, the transcription factor is selected from: EN2, CDX2 or TTF-1. In another embodiment, the transcription factor is CTCF.

Most of these transcription factors are not 100% tissue specific but may be expressed in a few cancers as well as a few adult tissue types. Detection of chromatin fragments containing the transcription factors in the blood are enhanced by use of analytically sensitive methods of detecting the associated DNA fragment(s). The disease and/or tissue specificity of the methods are enhanced by combining the identity of the transcription factor with the particular sequence(s) of DNA associated with it.

In one embodiment a body fluid sample taken from a subject is contacted with one or more transcription factor binding agents selected to test for one or more disease conditions in a multiplex assay. For example, testing for multiple transcription factors, each specific for one or more cancer diseases, optionally in addition to transcription factors expressed in many cancers, enables a test for the detection of many different cancer diseases in addition to identifying the tissue of the cancer in a single blood test. Methods for multiplex testing are well known in the art, for example, without limitation, the multiplex beads system of Luminex Corporation can be used to conduct large numbers of multiplexed assays in a single sample (Dunbar, 2006).

According to a further aspect of the invention, there is provided a method of detecting a disease in a human or animal subject which comprises the steps of:
(i) contacting a body fluid sample obtained from the human or animal subject with a plurality of binding agents which bind to a plurality of transcription factors;
(ii) analysing the DNA associated with the different transcription factors; and
(iii) using the presence and/or amount and/or pattern of DNA bound to a plurality of transcription factors to determine the presence and/or nature of the disease in the subject.

According to a further aspect of the invention, there is provided a method of detecting a disease in a human or animal subject which comprises the steps of:
(i) contacting a body fluid sample obtained from the human or animal subject with two or more (e.g. a plurality) of binding agents which bind to two or more (e.g. a plurality) transcription factors;
(ii) determining the sequence of the DNA associated with the transcription factors bound in step (i); and
(iii) using the presence and/or amount and/or pattern and/or sequence(s) of DNA bound to the transcription factors to determine the presence and/or nature of the disease in the subject.

In one embodiment each of a plurality of transcription factors is attached to a separate solid phase support so that each transcription factor can be isolated for analysis or sequencing of its associated DNA fragments. For example, the Luminex multiplex beads system consists of a multiplicity of bead types each of which may be coated with a different transcription factor binder which can be exposed to a single sample and subsequently isolated from each other for the (separate) sequencing of the DNA associated with each transcription factor independently.

### Transcription Factor-DNA Chromatin Fragments

Chromatin fragments present in the circulation originate from a variety of sources. One source is through release of chromatin into the circulation following the death of cells which may include diseased cells, for example cancer cells. In some cases there may be an active release of chromatin into the circulation.

A major source of chromatin fragments in the circulation is derived from neutrophils, through production of neutrophil extracellular traps (NETs) by a process known as NETosis. In this process neutrophils eject chromatin material (NETs) into the extracellular matrix to trap and neutralize pathogens locally to a site of infection. NETs and their metabolites are comprised largely of oligonucleosomes and mononucleosomes with component DNA fragments of sizes ≥150 bp.

Size profiling of cfDNA extracted from the blood reveals that the major component of cfDNA is mononucleosomes with a size distribution peak around 160-170 bp ranging from around 130-200 bp corresponding to mononucleosomes with varying lengths of associated linker DNA. There may be further peaks corresponding to various sizes of oligonucleosomes including, for example di-nucleosomes (around 340 bp), tri-nucleosomes (510 bp) and so on. In samples affected by NETosis there may also be broad peaks relating to large chromatin fragments ranging up to several thousand bp in length.

Transcription factors bind to short DNA sequences and transcription factor-DNA complexes contain much shorter DNA fragments in the range 35-80 bp (Snyder et al, 2016). In a typical size profile diagram of a double-stranded plasma cfDNA library, there is little or no material visible corresponding to cfDNA fragment lengths <100 bp in length. However, single stranded library preparations contain more cfDNA fragments in the 35-80 bp range (Snyder et al, 2016). This protein bound 35-80 bp cfDNA component is a minor component of total circulating chromatin fragments.

A further important aspect of transcription factor-DNA binding in the context of the present invention relates to the kinetic stability of the transcription factor-DNA binding. Some transcription factors are stably bound in vivo to DNA at a TFBS. Other transcription factors are transiently bound in vivo at a TFBS where they associate, disassociate and reassociate in a dynamic manner. In ChIP-Seq methods using cellular and tissue based substrates this is not an issue because both may be detected using cross-linking techniques. Dynamically bound transcription factors alternate naturally between bound and free forms, but when cross linked they become “trapped” in a bound form. Therefore, the use of short cross-linking times leads to high detection of stably bound transcription factors but less detection of dynamically bound transcription factors. In contrast, the use of longer cross-linking times leads to an increased detection of dynamically bound transcription factors as more become “trapped” in associated form by cross-linking over time (Poorey et al, 2013).

However, based on kinetic considerations, we reasoned that dynamically bound transcription factors are unlikely to be present in the circulation in blood or in other body fluids. In vivo there is a relatively high nuclear concentration of both chromatin and transcription factors allowing for association, disassociation and reassociation of the dynamically bound transcription factor-DNA complex. However, the level of a transcription factor-DNA complex in body fluids is highly diluted and present at so low a concentration that once disassociated, any transiently or dynamically bound transcription factor and DNA components are unlikely to reassociate. We reasoned that cross-linking in plasma will therefore only be relevant for stably bound transcription factors and will therefore always be rapid (because the slower cross-linking transiently bound transcription factors will be disassociated and can be disregarded). Therefore, in one embodiment of the invention there is provided a method of detecting a disease in a human or animal subject which comprises the steps of:
(i) contacting a body fluid sample obtained from a human or animal subject with a binding agent which binds to a kinetically stable transcription factor-DNA complex;
(ii) determining the sequence of one or more DNA fragments associated with the transcription factor in said kinetically stable transcription factor-DNA complex; and
(iii) using the presence of the transcription factor and the sequence of the associated DNA as a combined biomarker for determining the presence and/or nature of the disease in the subject.

### Transcription Factor DNA Binding Domains

Transcription factors may be classed according to their DNA Binding Domain (DBD). Vaquerizas et al, 2009 investigated 1391 known transcription factors and identified more than 24 different types of transcription factor based on DBD. The most commonly occurring transcription factors identified were those with a zinc finger DBD and these accounted for almost half (48.5%) of all transcription factors.

The preferred sample type for analysis of cfDNA, ctDNA or nucleosomes is EDTA plasma. The function of EDTA or citrate in a plasma blood collection tube is to chelate and sequester calcium ions in the blood to prevent clotting (the clotting cascade in blood requires the presence of calcium ions). Centrifugation of the tube separates the cellular component of the blood from the plasma supernatant, which can be removed and used as a sample matrix for many clinical diagnostic purposes.

The binding of zinc finger transcription factors to their DNA TFBS is dependent on the presence of zinc ions. However, the calcium chelators used in plasma blood collection tubes also chelate zinc ions. Chelation and removal of zinc ions from a zinc finger transcription factor may result in a loss of transcription factor binding to DNA (Ralston, 2008). The interaction of zinc chelating agents and zinc finger transcription factors means this family of transcription factors behave differently in EDTA plasma than transcription factors that bind DNA through other DBD types.

The presence of zinc finger transcription factor-DNA complexes in the blood has not been directly demonstrated. We reasoned that whilst such complexes exist, they have not been isolated because they are a small fraction of the small circulating chromatin fragment component of the blood (the vast majority of circulating chromatin fragments are nucleosomes) and, moreover, are disassociated in the plasma samples used by workers in the field. As described herein, we have addressed these two problems and demonstrated plasma ChIP-Seq of CTCF which is a zinc finger transcription factor.

### Transcription Factor Binding Agents

Preferred transcription factor binding agents include antibodies directed to bind to the transcription factor, or oligonucleotides, such as the DNA sequence of a TFBS (optionally including flanking sequences). Preferred binding agents have high affinity for the transcription factor, so that binding will occur at low transcription factor concentrations, as well as high specificity for binding of the transcription factor, so that non-specific binding of other proteins is minimal.

The binding agent may be coated on a solid support, such as sepharose, sephadex, plastic or magnetic beads. In one embodiment, said solid support comprises a porous material. In another embodiment, the binding agent is derivatized to include a tag or linker which can be used to attach the binding agent to a suitable support which has been derivatized to bind to the tag. Many such tags and supports are known in the art (e.g. Sortag, Click Chemistry, biotin/streptavidin, his-tag/nickel or cobalt, GST-tag/GSH, antibody/epitope tags and many more). Isolation of the binding agent may then be performed prior to, concurrently with, or following the reaction of the binding agent with a transcription factor. For ease of use, the coated support may be included within a device, for example a microfluidic device. Multiple solid phase binding agents may be used in a multiplex assay format for the simultaneous testing for the presence of multiple chromatin fragments containing different transcription factors, in a single test in a single body fluid sample.

In other embodiments the binding agent is added in solution and isolated by cross-linking and precipitating the bound nucleosomes with a precipitation agent such as polyethylene glycol (PEG). The precipitated pellet can then be isolated as a separate phase, for example by centrifugation or filtration. Many immunoprecipitation methods are known in the art and any such methods may be useful in methods of the invention.

In some embodiments, the DNA associated with the transcription factor is bound by a DNA binding agent. The DNA binding agent may be attached to a solid phase (for example plastic particles, magnetic particles, agarose or many others.) The DNA binding agent may be directly or indirectly (for example, through a linker system such biotin/avidin or glutathione) attached to the solid phase.

We have used commercially available antibodies directed to bind to transcription factors. For ChIP-Seq we immobilized the antibodies on commercially available magnetic polystyrene particles. Therefore in a preferred embodiment of the invention the transcription factor binding agent is a solid phase anti-transcription factor antibody (or a part thereof) immobilized on a magnetic polystyrene particle.

### DNA Library Preparation

Some embodiments of the present invention include the preparation of a library of cfDNA fragments associated with transcription factors in chromatin fragments. A library may be amplified for ease of detection and sequencing using PCR methods. In principle any library preparation method may be suitable for use with methods of the invention.

DNA fragment library preparation methods are well known in the art and typically involve the ligation of adapter oligonucleotides to the DNA fragments. Amplification of the adapter ligated DNA fragment library is typically performed by PCR. PCR primers may also be used for DNA amplification and may be degenerate to amplify all sequences present in a library, or may be designed using software known in the art to amplify specific DNA sequences associated with the sequence of a response element of a transcription factor optionally also including flanking regions.

Library preparation methods may involve single-stranded or double-stranded adapter ligation of cfDNA fragments. Preferred library preparation methods involve single-stranded cfDNA adapter ligation. Preferred library preparation methods have high efficiency for amplification and isolation of small DNA fragments of less than 100 bp in length. Many such library preparation methods are known in the art including for example, (i) the TruSeq DNA Sample preparation Kit (Illumina) used according to the manufacturer's protocol with 20-25 PCR cycles for 5-10 ng of input DNA (Ulz et al, 2019), (ii) use of the MagMAX cfDNA Isolation Kit (Applied Biosystems) followed by library preparation using the NEBNext Ultra II DNA Library Prep Kit (New England Biolabs) (Ulz et al, 2019) or (iii) use of the blood and body fluid protocol for the Qiagen QIAamp DSP DNA Blood Mini Kit with PCR amplification using the Life technologies Ion Plus Fragment Library Kit (Hu et al, 2019). Other methods include those described by Sanchez et al, 2018, Skene and Henikoff, 2017, Snyder et al, 2016 and Liu et al, 2019. In the Examples provided herein, we used a commercially available single strand DNA library preparation kit (Claret Bio SRSLY NGS Library Prep Kit).

It will be clear to those skilled in the art that for embodiments of the invention where PCR amplification of the transcription factor associated DNA is performed (only) to increase the sensitivity of transcription factor detection or quantitation, then the amplification of the response element sequence alone, without flanking sequences, is sufficient.

### Immunoprecipitation of Transcription Factor-DNA Complexes

Immunoprecipitation is in principle a simple process. In a typical method, an antibody directed to bind specifically to a protein of interest is coated to a solid support and exposed to a biological sample containing the protein. The protein of interest is bound by the antibody and hence adsorbed to the surface of the solid phase, whilst other proteins and other substances remain in solution. The solid phase is isolated from the sample and washed leaving a pure sample of the protein of interest attached to the solid support.

Cell and tissue based ChIP-Seq methods are well described in the art. It is usual to use 20-30 μg of digested or sonicated chromatin extracted from tissue or cultured cells as the substrate material. As chromatin consists of approximately 40% DNA this represents some 8-24 μg of substrate DNA. However, the concentration of circulating cfDNA is low and has been measured at 30±14 ng/ml in healthy human subjects and 71±55 ng/ml in gastric cancer patients (Park et al, 2012). Therefore a 1 ml plasma sample will yield approximately 200-500 fold less chromatin material than normally used in ChIP-Seq.

As the majority of circulating cell free chromatin is comprised of nucleosomes, the available circulating cell free transcription factor-DNA chromatin fragment material is extremely small. Moreover, the available circulating cell free transcription factor-DNA chromatin fragment material will comprise thousands of transcription factors. Therefore, the available substrate material for analysis by methods of the invention represented by a single transcription factor will be a small fraction of the small amount of circulating cell free transcription factor-DNA material present in the circulation.

In addition, chromatin extracts from cells are relatively pure chromatin material. In contrast body fluids, such as blood, serum or plasma contain a small amount of chromatin but higher concentrations of a huge number of proteins and other compounds any of which may interfere in methods of the invention by adhering non-specifically to the solid phase transcription factor antibody or other binding agent used. An extra complexity for the immunoprecipitation of circulating transcription factor DNA complexes from blood, serum or plasma is that the background non-specific binding is therefore high in relation to the small amount of target transcription factor bound to a specific binder on a solid phase support and may obscure its detection.

Due to all these difficulties, there are few reports in the literature of ChIP-Seq in plasma or other blood sample matrices. Where ChIP-Seq in plasma has been described it has been for nucleosomes and nucleosomal histones as the level of these is high (in relation to the level of a single transcription factor).

We have addressed these difficulties by using high avidity antibodies and by reducing the non-specific binding of other proteins on the solid phase support to extremely low levels through the use of an appropriate solid phase support combined with stringent washing of the solid phase with solutions containing high concentrations of strong detergents.

Therefore, the antibody bound transcription factor-DNA complex may be washed with a strong (e.g. a concentration of at least 1%, for example 1.2%) detergent or mix of detergents prior to extraction of the transcription factor associated DNA. In one embodiment, the transcription factor bound by the binding agent in step (i) is washed with a buffer solution containing at least 1% concentration of detergent, prior to detection of the associated DNA fragment. There are a very large number of detergents that may be used for this purpose. A few common examples include, without limitation, Triton detergents (e.g. Triton X-100), Tween detergents (e.g. Tween 20 and Tween 80), sodium deoxycholate, sodium dodecyl sulfate, octylphenoxypolyethoxyethanol (IGEPAL CA-630), tricosaethylene glycol dodecyl ether (Brij), n-dodecyl-beta-maltososide, octyl-beta-glucoside, octylthio glucoside, 3-((3-cholamidopropyl) dimethylammonio)-1-propanesulfonate (CHAPS) and many more.

We used magnetic polystyrene microbeads and washed repeatedly (5 washes) with a wash solution containing a mixture of 1% octylphenoxypolyethoxyethanol, 0.1% sodium deoxycholate and 0.1% sodium dodecyl sulfate.

In a preferred embodiment of the invention the solid-phase support is a polystyrene particle, for example a magnetic polystyrene particle. The antibody (or other binder of a transcription factor) used may be directly or indirectly attached to the support.

In a preferred embodiment of the invention the solid phase bound transcription factor-DNA complex isolated on the solid-phase support is washed with a solution containing at least 0.25%, or at least 0.5% or at least 1% of detergent or surfactant. The detergent used may consist of a single detergent or of a mixture of detergents as described herein.

In one embodiment of the invention, the solid phase transcription factor binder support used comprises a multiplexed system, for example a multiplexed bead system (such as the system provided by Luminex Corporation). In this solid support system multiple beads, which can be distinguished on the basis of fluorescence, may each be coated with a different specific binder for a different transcription factor and used simultaneously to investigate multiple transcription-factor-DNA complexes in a single sample (Dunbar, 2006).

### DNA Sequencing

There are many methods known in the art to analyze, quantify or identify a DNA sequence and any DNA analysis method may be employed for methods of the current invention including, without limitation, next generation sequencing methods, isothermal DNA amplification, cold PCR (co-amplification at lower denaturation temperature-PCR), MAP (MIDI-Activated Pyrophosphorolysis), PARE (personalized analysis of rearranged ends), DNA hybridization methods (including gene chip methods and in situ hybridization methods). In addition, the gene sequence may also be analyzed for epigenetically altered DNA sequences by epigenetic DNA sequencing analysis (e.g. for sequences containing 5-methylcytosine using bisulfite conversion of unmodified cytosine to uracil). Therefore, in one embodiment, the associated DNA is analyzed using DNA sequencing, for example a sequencing method selected from Next Generation Sequencing (targeted or whole genome) and methylated DNA sequencing analysis, BEAMing, PCR including digital PCR and cold PCR (co-amplification at lower denaturation temperature-PCR), isothermal amplification, hybridization, MIDI-Activated Pyrophosphorolysis (MAP) or Personalized Analysis of Re-arranged Ends (PARE).

The Examples described herein have used Illumina NovaSeq sequencing. Therefore, in a preferred embodiment of the invention the DNA that is extracted from the isolated transcription factor is analysed by next generation sequencing.

### Sample Preparation

The sample may be any body fluid in which chromatin fragments can be detected. Chromatin fragments are known to occur in blood, feces, urine and cerebrospinal fluid. We have also detected chromatin fragments in sputum. In preferred embodiments, the body fluid sample is a blood, serum or plasma sample. These samples may be used to measure and analyze circulating cell free chromatin fragments containing a transcription factor and a fragment of DNA.

Where blood samples are used for methods of the invention this may be whole blood, a serum sample or a plasma sample. Whole blood or serum samples may be used as substrates for the analysis of any (stably-bound) transcription factor-DNA chromatin fragment involving a transcription factor with any DBD type.

Plasma samples, such as EDTA plasma samples may also be used in methods of the invention. In a typical plasma sample collection method, the whole blood is collected into a citrate or EDTA blood collection tube and centrifuged within 2 hours. The resulting supernatant plasma may be used fresh or may be frozen until analyzed. However, calcium ion sequestrators used as additives to blood collection tubes to produce plasma, cause disassociation of circulating zinc finger transcription factor-DNA complexes. As noted above, the most common class of transcription factors are the zinc finger transcription factors.

There are a number of ways to overcome this difficulty including, without limitation: (i) to avoid using zinc finger transcription factors and use transcription factors with other DBD types, (ii) to use serum samples, (iii) to use heparin plasma or other plasma sample types not involving calcium sequestration or (iv) to prevent the disassociation of transcription factor-DNA complexes, for example by cross linking the proteins and/or DNA in the chromatin fragment in a blood sample.

In one embodiment the body fluid sample is a serum sample. Serum is thought to contain contaminating chromatin material derived from white blood cells (e.g. NETs). This contamination interferes in the analysis of cfDNA and therefore plasma is the sample matrix most commonly used for ctDNA methods. However, the isolation of a chromatin fragment containing a transcription factor from other chromatin material present prior to DNA analysis removes such interference. Moreover, the contamination of serum with chromatin material is a result of the formation of neutrophil extracellular traps (NETs) by neutrophil cells in the blood sample triggered by coagulation (a known inducer of NETosis). Provided the serum sample collection tube containing whole blood is processed in a timely manner, for example 15-60 minutes following venipuncture, the contaminating NETs material will be large chromatin rather than small chromatin fragments and will not interfere in the analysis of small transcription factor-DNA complexes. Therefore widening the sample type that may be used is a further advantage of the methods of the invention.

The presence of contaminating NETs in serum may be further minimized or eliminated by the addition of an inhibitor of NETosis to the serum blood collection tube. This prevents NETosis and hence minimizes the level of background chromatin present in a serum sample. Many inhibitors of NETosis are known in the art. Preferred inhibitors include the anthracycline class of drugs, in particular doxorubicin. Therefore, in one embodiment of the invention, there is provided a method of detecting a cell free chromatin fragment comprising a transcription factor and a DNA fragment in a serum sample obtained from a human or animal subject which comprises the steps of:
(i) obtaining a whole blood sample from a subject in a serum blood collection tube;
(ii) contacting the whole blood sample with an inhibitor of NETosis;
(iii) isolating a sample of serum from the whole blood sample;
(iv) contacting the serum sample with a binding agent which binds to a transcription factor;
(v) detecting or measuring the DNA fragment associated with the transcription factor; and
(vi) using the presence or amount or sequence of the DNA fragment as a measure of the amount of cell free chromatin fragments comprising the transcription factor in the serum sample.

It will be understood that this embodiment of the invention may also be used to provide information as an indicator of the disease state of a subject, as described previously herein.

In one embodiment, the body fluid sample is any plasma sample including a plasma sample produced using a calcium sequestrator such as EDTA plasma or citrate plasma wherein the plasma sample is obtained by contacting a whole blood sample with a cross-linking agent. The cross-linking agent may be contacted with whole blood in a first step of a process involving: (1) contacting a whole blood sample with a cross-linking agent; (2) contacting the cross-linked sample with a calcium ion chelating agent; and (3) isolating plasma from the sample.

Cross linking is a well known technique in the art. The most commonly used cross linking reagent is formaldehyde which binds protein molecules to each other and to DNA. However, excess cross linking may lead to changes in the structure of antibody binding epitopes in transcription factors (and hence to loss of antibody binding) and even the cross linking of transcription factors to separate protein molecules or complexes. To prevent this, cross linking is often quenched a few seconds or minutes after adding formaldehyde, for example by addition of excess glycine or tris(hydroxymethyl)aminomethane (TRIS), to stop further cross linking. Therefore, in one aspect of the invention, there is provided a method of detecting, analysing or measuring a chromatin fragment containing a transcription factor and associated DNA fragment in a blood sample taken from a human or animal subject which comprises the steps of:
(i) contacting the blood sample obtained from the subject with a cross linking agent;
(ii) optionally adding a quenching agent to stop further cross linking;
(iii) contacting the sample with a calcium ion chelating agent;
(iv) isolating plasma from the sample;
(v) contacting the plasma sample with a binding agent which binds to a transcription factor;
(vi) isolating bound chromatin fragments containing the transcription factor; and
(vii) analysing the isolated chromatin fragments (e.g. by methods as described herein).

In another aspect of the invention, there is provided a method of detecting a disease in a human or animal subject which comprises the steps of:
(i) contacting a blood sample obtained from the subject with a cross linking reagent;
(ii) optionally adding a quenching agent to stop further cross linking;
(iii) contacting the sample with a calcium ion chelating agent;
(iv) isolating plasma from the sample;
(v) contacting the plasma sample with a binding agent which binds to a transcription factor;
(vi) isolating the DNA associated with the transcription factor;
(vii) optionally amplifying the isolated DNA by a PCR method;
(viii) determining the amount and/or the sequence of the DNA; and
(ix) using the presence of the transcription factor and/or the sequence of the associated DNA as a biomarker for detecting the disease status of the subject.

In a preferred embodiment, formaldehyde or a formaldehyde releasing agent is used as a cross linking agent. In one embodiment, EDTA is used as a chelator of calcium ions to prevent coagulation. In preferred embodiments the formaldehyde is added to whole blood immediately following the collection of the whole blood sample, for example by adding the whole blood sample to a tube already containing formaldehyde. The tube is left for sufficient time for the cross linking reaction to proceed and then the reaction is stopped by the addition of a quencher to prevent excess cross linking of plasma components. The quencher is typically an amine compound such as glycine or TRIS that reacts with formaldehyde. The quencher may be added with the EDTA, for example by addition of a solution of glycine and EDTA in TRIS buffer. The whole blood sample is then centrifuged and the plasma containing cross linked transcription factor bound DNA complexes, is isolated for analysis by methods of the invention.

In another aspect of the invention, there is provided a method of detecting a disease in a human or animal subject which comprises the steps of:
(i) contacting a blood sample obtained from the human or animal subject with a cross linking reagent;
(ii) contacting the whole blood sample with a quenching reagent and a calcium ion chelating agent;
(iii) isolating the plasma produced from the sample in step (ii);
(iv) contacting the plasma sample with a binding agent which binds to a transcription factor;
(v) isolating the DNA associated with the transcription factor;
(vi) optionally amplifying the isolated DNA;
(vii) determining the amount and/or the sequence of the DNA; and
(viii) using the presence of the transcription factor and/or the sequence of the associated DNA as a biomarker for detecting the presence and/or the nature of a disease in the subject.

As discussed above, the transcription factors present in the circulation are most likely those that are stably bound to DNA rather than those which associate transiently with DNA and disassociate in a dynamic manner. For most stably bound DNA circulating chromatin fragments including transcription factors, cross linking with formaldehyde in whole cultured cells or tissue samples is rapid and takes less than 1 or 2 minutes. We reasoned that, whilst 1 or 2 minutes may be required for diffusion and entry of formaldehyde into a cell, followed by entry into the nucleus, followed by cross-linking of chromatin, this time may be reduced in a whole blood context where the chromatin fragments are free in solution and immediately accessible to cross-linking. The cross linking reagent used may be formaldehyde or be a formaldehyde releasing agent (also called a formaldehyde releaser, formaldehyde donor or formaldehyde releasing preservative). A formaldehyde releasing agent is a moiety that slowly releases formaldehyde. Many formaldehyde releasing agents are known in the art and are commonly used as antimicrobial preservatives in the cosmetics industry, for example in skin care and hair care products where high levels of formaldehyde are avoided due to toxicity but low protective levels are maintained by release. Therefore, in one embodiment the cross linking agent is a formaldehyde releasing agent.

We reasoned that cross-linking of cell free circulating transcription factor-DNA complexes in whole blood (as opposed to cells or tissue) is rapid and may occur more rapidly than zinc depletion of the zinc finger proteins. Therefore, in one embodiment of the invention, the cross linking reagent may be added simultaneously with the calcium ion chelator. Blood collection tubes (BCT) containing both EDTA and a formaldehyde releasing agent are available commercially, for example the Cell-Free DNA BCT available from Streck Inc. Whole blood added to such tubes is exposed simultaneously to EDTA and a cross-linking agent.

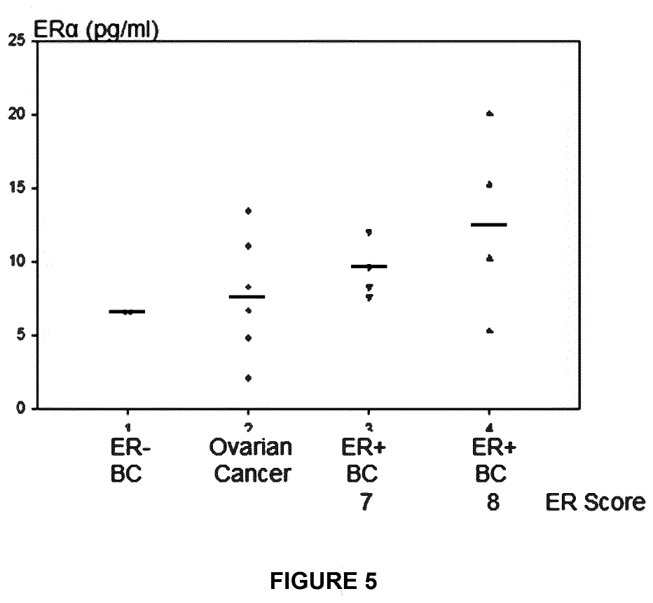

We performed a number of experiments using different EDTA sample preparation methods. For example, the Estrogen Receptor (ER) is a zinc finger transcription factor. We measured the level of ER present in (regular) EDTA plasma samples using an ELISA method. ER was detectable as shown in FIG. 5. We immunoprecipitated ER from EDTA plasma samples, extracted DNA bound to the solid phase and amplified DNA present in the extract. However, no DNA was observed in the amplified samples. We reasoned that this was because ER-DNA complexes were disassociated in EDTA plasma.

CTCF (also called CCCTC-binding factor) is an evolutionarily conserved zinc finger transcription factor that binds through a combination of 11 zinc fingers to a large number of sites in the genome and has a critical role in genome function. An investigation of CTCF binding sites in the human genome identified 77,811 distinct binding sites across 19 different cell types (Wang et al, 2012). 27,662 of the 77,811 binding sites were found to be occupied in all 19 cell types investigated. CTCF binding of the remaining 50, 149 binding sites exhibited tissue specificity. The 19 cell types investigated included 12 normal cell types and 7 cancer or EBV-immortalised cell lines representing colorectal cancer (Caco-2), cervical cancer (HeLa—S3), hepatocellular cancer (HepG2), neuroblastoma (SK—N-SH_RA), retinoblastoma (WERI-RB-1) and EBV-transformed lymphoplastoid (GM06990). CTCF binding at 1,236 binding sites was found to be specific to cancer cell lines, and occupancy of these binding sites distinguished immortal and cancer cell lines from normal cells including epithelia, fibroblasts and endothelia (Liu et al, 2017).

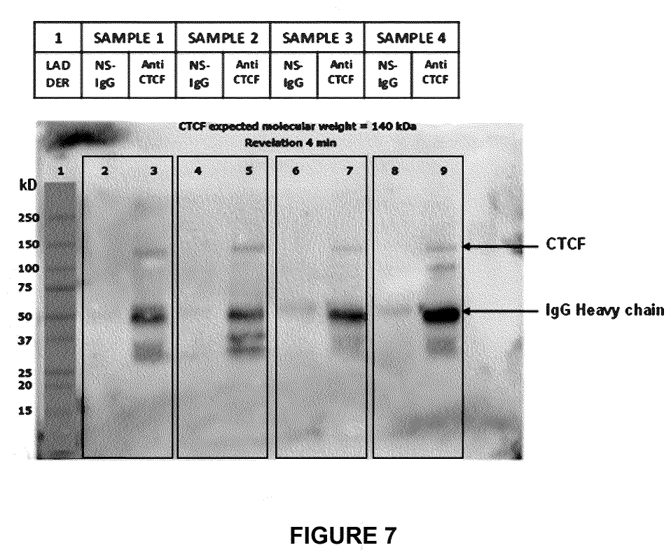

We used a mouse anti-CTCF antibody to immunoprecipitate CTCF-DNA from 4 pooled cross-linked EDTA plasma samples (collected in Streck cfDNA BCTs) collected from 18 subjects diagnosed as suffering from cancer. We performed Western blot analysis of the protein isolated by ChIP on the solid phase support. The results in FIG. 7 show that a protein band corresponding to CTCF at a molecular weight of approximately 140 kD was present in all 4 pooled samples (but not in control experiments employing non-specific mouse IgG in place of the anti-CTCF antibody). The band at approximately 50 kD corresponds to binding of the labelled anti-mouse IgG antibody used for Western blot to the heavy chain of the mouse anti-CTCF antibody employed for ChIP.

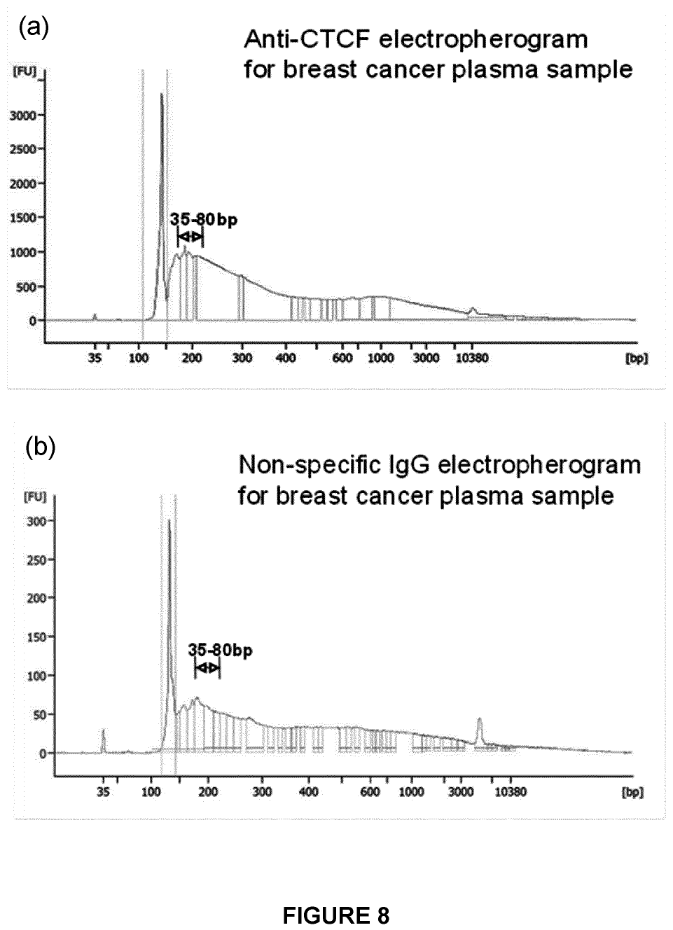

We then repeated the ChIP method to immunoprecipitate CTCF-DNA complexes using cross-linked EDTA plasma samples (collected in Streck cfDNA BCTs) collected from a subject diagnosed as suffering from breast cancer. We extracted cfDNA fragments from the solid phase support, ligated the extracted DNA fragments to adapter oligonucleotides and amplified the cfDNA present. The amplified cfDNA library was analysed by electrophoresis and the resulting electropherograms (FIG. 8) showed that the library contained small fragments in the 35-80 bp range (which correspond to a peak between 175-220 bp on the x-axis to account for the adapter ligated fragments). The major peak of adapter ligated cfDNA fragments was observed at approximately 50 bp in length (which corresponds to a peak at 190 bp on the x-axis to account for the adapter ligated fragment length). Although, the amplified cfDNA library contained small fragments in the 35-80 bp range, not all these fragments were bound to CTCF in the sample because small DNA fragments were also obtained for amplified extracts from solid supports coated with non-specific mouse IgG. However, the specific peak obtained with specific anti-CTCF antibody ChIP (1000 fluorescence units (FU)) was higher than the non-specific IgG peak (80 FU).

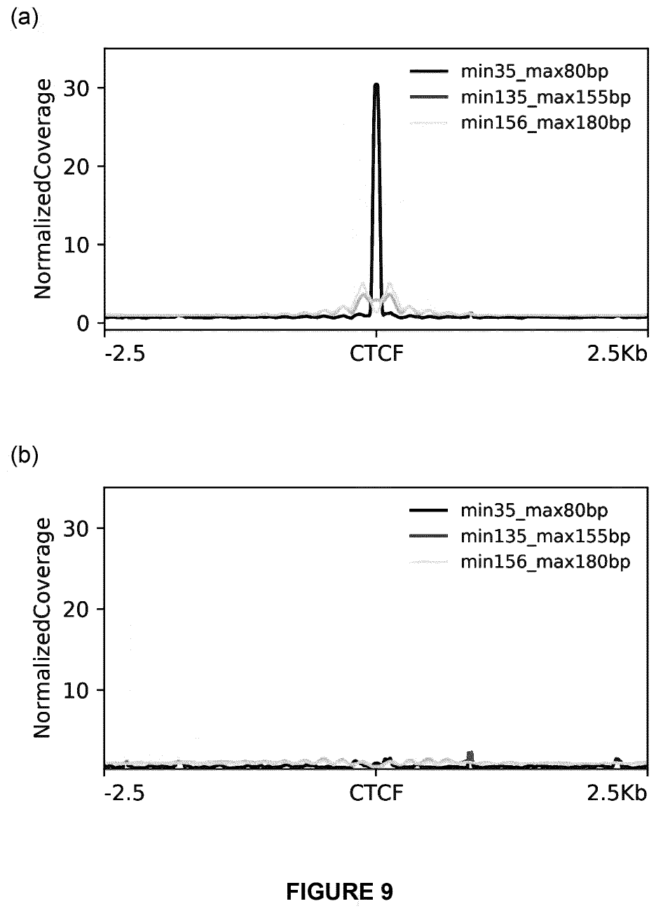

The amplified cfDNA library isolated using anti-CTCF immunoprecipitation was sequenced by Next Generation sequencing methods. Results for an amplified library prepared from a cross-linked EDTA plasma sample (collected in Streck cfDNA BCTs) collected from a patient diagnosed with CRC are shown in FIG. 9. We observed enrichment of small cfDNA fragment binding of 9780 published CTCF TFBS sequences (Kelly et al, 2012). By contrast, the cfDNA library obtained for binding to non-specific mouse IgG showed no enrichment. Peak calling of the cfDNA fragment sequences with reference to the input non-specific control resulted in CTCF as the transcription factor with the most TFBS sequence fragments. We conclude that the methods of the invention are successful for the ChIP-Seq of transcription factors in plasma.

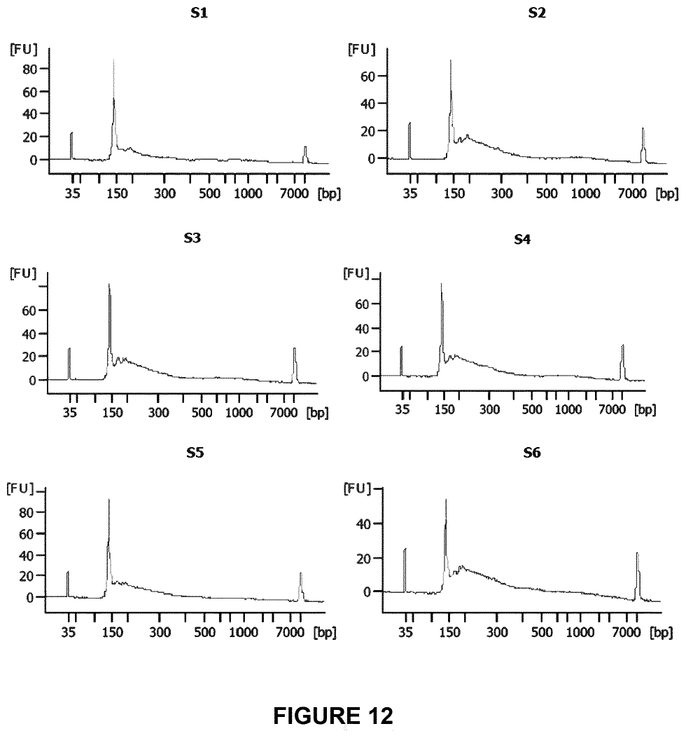

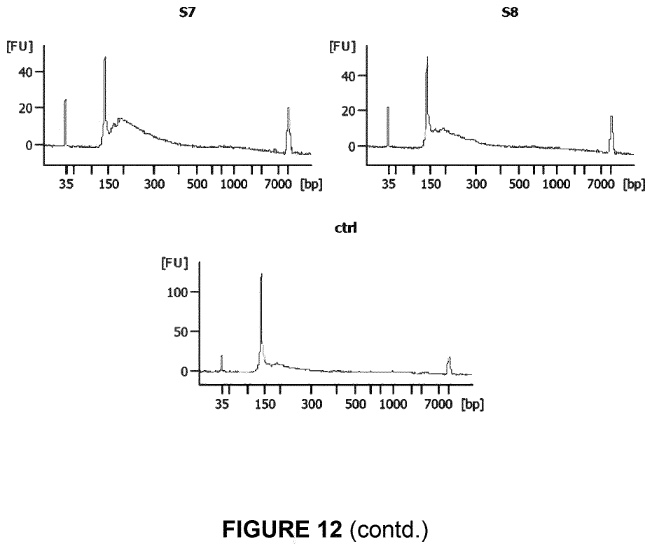

The androgen receptor (AR) is a zinc finger transcription factor of interest in prostate cancer. To show that the method of the invention may be applied to a less abundant transcription factor than CTCF, we applied the same method to AR. We used a mouse anti-AR antibody to immunoprecipitate AR from cross-linked EDTA plasma samples (collected in Streck cfDNA BCTs) from 8 subjects diagnosed as suffering from prostate cancer. We performed Western blot analysis of the protein isolated by ChIP on the solid phase support using AR from LnCAP prostate cancer cell line cells as a positive control. The results in FIG. 11 show that a protein band corresponding to AR at a molecular weight of approximately 10 kD was present in all 8 samples and particularly strong in 2 samples (lanes 2 and 3 of FIG. 11). The band at approximately 50 kD corresponds to binding of the labelled anti-mouse IgG antibody to the heavy chain of the mouse anti-AR antibody employed for ChIP. We then extracted DNA from the solid phase supports, ligated the extracted DNA fragments to adapter oligonucleotides and amplified the DNA present. The results in FIG. 12 show that the amplified cfDNA library contained small fragments in the 35-80 bp range (as above, a peak shown at 175-220 bp for adapter linked fragments). Although, the amplified cfDNA library contained small fragments in the 35-80 bp range, not all these fragments were bound to AR in the sample because small DNA fragments were also obtained for amplified extracts from solid supports coated with non-specific mouse IgG. The amplified cfDNA libraries obtained for the 2 samples with the highest observed levels of AR by Western were then sequenced by Next Generation Sequencing.

### Disassociated Transcription Factor-DNA Complexes

The previous aspects of the invention are methods to detect, measure or characterise a chromatin fragment including a transcription factor bound directly or indirectly to DNA. In one embodiment of the invention there is a method for the detection of a transcription factor that is not DNA bound (i.e. a free or unbound transcription factor) in a body fluid sample taken from a subject. Detection of free transcription factor may be performed by using an oligonucleotide including the TFBS DNA sequence of the transcription factor, optionally including flanking sequences, as a binding agent for the free transcription factor. Oligonucleotide bound free transcription factor may then be detected, for example using a labelled anti-transcription factor antibody (e.g. see Active Motif, 2006). Transcription factors may initially be produced in an inactive form which may later be post-translationally activated, for example by phosphorylation. Active transcription factor forms bind to an oligonucleotide that includes their TFBS sequence. Inactive transcription factor forms do not bind to an oligonucleotide that includes their TFBS sequence (Lee et al, 2007). Thus, active, free transcription factor may be detected in a body fluid sample using an assay involving the binding of the free transcription factor to an oligonucleotide including a DNA sequence to which the transcription factor binds, for example a TFBS sequence of the transcription factor, followed by addition of a second transcription factor binding agent, for example an anti-transcription factor antibody directed to bind specifically to the transcription factor and using the presence or degree of antibody binding as a measure of the presence or amount of active free transcription factor present in the sample. Therefore, in one embodiment of the invention there is provided a method of detecting a free transcription factor in a human or animal subject which comprises the steps of:
(i) contacting a body fluid sample obtained from the human or animal subject with an oligonucleotide which binds to a transcription factor;
(ii) isolating the oligonucleotide bound transcription factor;
(iii) contacting the isolated transcription factor with a second binding agent which binds to the transcription factor; and
(iv) using the presence or degree of the second binding agent to the transcription factor as a measure of the amount of cell free transcription factor in the sample.

In preferred embodiments the oligonucleotide used to bind to the free transcription factor includes a TFBS sequence. In preferred embodiments the oligonucleotide used to bind to the free transcription factor is attached to a solid phase support. In preferred embodiments the second binding agent is an antibody. In preferred embodiments the second binding agent is labelled so that its binding to the solid phase oligonucleotide bound transcription factor can be readily detected and/or quantified.

In one embodiment zinc ions are added to the sample to facilitate the binding of oligonucleotides to zinc finger transcription factors. Zinc ions may be added simultaneously with the addition of the oligonucleotide in step (i), or prior to step (i).

In another aspect of the invention, there is provided a method of detecting a disease in a human or animal subject which comprises the steps of:
(i) contacting a body fluid sample obtained from the human or animal subject with an oligonucleotide which binds to a transcription factor;
(ii) isolating the oligonucleotide bound transcription factor;
(iii) contacting the isolated transcription factor with a second binding agent which binds to the transcription factor; and
(iv) using the presence or degree of binding of the second binding agent to the transcription factor as an indicator of the presence and/or the nature of a disease in the subject.

In one embodiment a body fluid sample taken from a subject is contacted with one or more oligonucleotides (for example, TFBS sequences specific for binding to one or more transcription factors) to identify the presence and/or nature of a disease. In a further embodiment, the method is performed using a multiplex assay (i.e. comprising more than one oligonucleotide, preferably wherein each oligonucleotide is specific for a different transcription factor) to test for one or more diseases. For example, testing for multiple transcription factors each specific for one or more cancer diseases, optionally in addition to transcription factors expressed in many cancers, enables a test for the detection of many different cancer diseases in addition to identifying the tissue of the cancer in a single blood test. Methods for multiplex testing are well known in the art, for example, without limitation, DNA microarray methods or the multiplex beads system of Luminex Corporation which can be used to conduct large numbers of multiplexed assays in a single sample (Dunbar, 2006).

In preferred embodiments, the disease is cancer. In a further embodiment, the nature of the disease is the tissue affected by the cancer.

The estrogen receptor (ER) is a ligand-activated nuclear hormone receptor zinc finger transcription factor. We reasoned that circulating chromatin fragments in the blood that include zinc finger transcription factors and a DNA fragment are likely to be disrupted in EDTA plasma samples. We performed enzyme linked immunosorbent assay (ELISA) measurements for free (i.e. not bound to DNA) Estrogen Receptor alpha (ERα) in plasma samples taken from patients with gynecological cancers that involve over-expression of the estrogen receptor, as well as from patients with ER-negative breast cancer. ER is involved in the regulation of the transcription of a large number of genes and is highly expressed in female reproductive tissues and reproductive cancer tissues. ER is expressed at low levels in hematopoietic cells but is highly expressed in ER-positive breast cancer and ovarian cancer cells. ER-positive cancer cells have estrogen receptors, are sensitive to estrogen and their growth is stimulated by estrogen. ER-negative cancer cells do not have estrogen receptors and are insensitive to estrogen. About 80% of ovarian and breast cancers are ER-positive. ER-positive cancer is associated with a better prognosis than ER-negative cancer. As ER-positive cancers grow in response to estrogen, they are amenable to hormone therapy including tamoxifen and aromatase inhibitors which inhibit activation of the estrogen receptor by binding to estrogen and hence prevent cancer growth.

The ER-positive or negative status of a cancer is determined by immunohistochemistry tests of surgically removed cancer tissue. Typically, a labelled antibody that binds to ER is incubated with cancer cells/tissue and the level of antibody staining observed determines the status. ER-positive cancers are assigned an ER score. The proportion of cancer cells that test positive for hormone receptors as well as the intensity of the staining are measured. The two parameters are combined to score the sample on a scale from 0 to 8. Samples with more receptors that are visible at higher intensity are scored higher.

As nuclear hormone receptors are cellular proteins, ER would not be expected to be present in the circulation. We hypothesized that any free ERα present in plasma must originate from circulating chromatin fragments that included ERα, but which dissociated to release free ERα from DNA binding on addition of EDTA to make plasma. We expected the levels of such chromatin fragments to be vanishingly low and hence expected to find that the level of free ERα in plasma would be undetectable to an ELISA method and below the minimal sensitivity of the ELISA used (0.8 pg/ml). Surprisingly, we found that free ERα is present in plasma at levels up to 20 pg/ml (FIG. 5). To put this in context, Interleukin-6 and Tumor Necrosis Factor are commonly measured blood biomarkers whose normal ranges are approximately 5-15 pg/ml and up to 8 pg/ml respectively. Moreover, the measured levels of ERα were higher in ovarian cancer and ER-positive breast cancer than in ER-negative breast cancer indicating a tumour origin for the ERα.

Therefore, in another aspect of the invention there is provided a method for the detection of the presence of, or the measurement of the level of, a zinc finger transcription factor in a biological sample which comprises the steps of:
(i) contacting the sample with a zinc ion chelating reagent; and
(ii) analysing the sample for the presence or level of the displaced zinc finger transcription factor.

In one embodiment the biological sample is a body fluid sample, such as blood, serum or plasma. In a further embodiment, the zinc ion chelating reagent is EDTA. The EDTA may be added to the body fluid sample to disrupt zinc finger-DNA binding.

In a preferred embodiment the biological sample is a whole blood sample and the zinc ion chelating reagent is EDTA which is added to the whole blood sample to disrupt zinc finger-DNA binding, as well as to prevent coagulation of the blood and hence produce a plasma sample containing free zinc finger transcription factor. Any method may be used for the analysis of the sample for a transcription factor. In a preferred embodiment the method of analysis employed is an immunoassay and particularly a 2-site “sandwich” immunoassay. Therefore, in a preferred embodiment of the invention there is provided a method for the detection of the presence of, or the measurement of the level of, a circulating chromatin fragment containing a zinc finger transcription factor in a whole blood sample taken from a subject which comprises the steps of:
(i) contacting the whole blood sample with EDTA to produce a plasma sample; and
(ii) analysing the plasma sample for the presence or level of the zinc finger transcription factor using an immunoassay method.

The zinc finger transcription factor family is the most abundant transcription factor family. Therefore, this aspect of the invention may be used to detect the majority of transcription factors of interest. The term “zinc finger transcription factor” refers to any transcription factor containing a zinc finger-binding domain.

The circulating zinc finger transcription factor may be used as a biomarker for the detection of disease, for example the detection, diagnosis, treatment selection, monitoring or prognosis of a gynecological cancer. Therefore, in one embodiment of the invention there is provided a method for the determination of the disease status of a subject, for example for the detection, diagnosis, treatment selection, monitoring or prognosis of or for the disease, in the subject which comprises the steps of:
(i) contacting a blood sample obtained from the subject with a zinc chelating agent to produce a plasma sample;
(ii) analysing the plasma sample for the presence or level of the zinc finger transcription factor; and
(iii) using the presence or level of the zinc finger transcription factor in the sample as an indicator of the disease status of the subject.

This aspect of the invention is also suitable for use with cell culture methods. Chromatin Immunoprecipitation (ChIP) methods for transcription factors are complex, difficult, time consuming and not robust. A typical ChIP method involves extraction of the chromatin material from a cell, fragmentation of the chromatin by DNA digestion or using a physical method such as sonication, isolation of chromatin fragments using an antibody, extraction of the DNA associated with the antibody and determining the DNA sequence of the extracted DNA. Using the method of the invention, the presence or amount of the zinc finger transcription factor may be established by extraction of the chromatin material from a cell into a fluid containing EDTA (or other zinc chelating agent) and measuring free zinc finger transcription factor (for example by ELISA).

Any method may be used for the analysis of the sample for the presence or amount of the zinc finger transcription factor including, without limitation, mass spectrometry and any immunochemical method. In preferred embodiments the method used for analysis of the sample for the presence or amount of the zinc finger transcription factor is an immunoassay.

As we have found that addition of zinc ion chelating agents to samples containing chromatin fragments including a zinc finger transcription factor results in the disruption of those chromatin fragments to produce free zinc finger transcription factors and EDTA is a strong chelator of zinc (as well as calcium) ions, it will be clear that zinc finger transcription factor bound DNA cannot be investigated in EDTA plasma samples using a method involving isolating the transcription factor with an antibody (or other transcription factor binder) and analysing the DNA associated with the transcription factor because the DNA will no longer be associated with the transcription factor.

It will be understood that disruption of a zinc finger transcription factor binding to DNA will lead to both free zinc finger transcription factor and also free DNA fragments including the sequence of the TFBS and flanking DNA sequences in the genome. Therefore, in a further aspect of the invention there is provided a method for identifying the presence of a circulating chromatin fragment containing a zinc finger transcription factor or the sequence(s) of DNA fragments bound to the zinc finger transcription factor in a subject which comprises the steps of:
(i) contacting a blood sample obtained from the subject with a zinc chelating agent to produce a plasma sample; and
(ii) analysing the plasma sample for the presence or level of free DNA fragments containing a DNA sequence including a transcription factor binding site sequence or a flanking sequence to a zinc finger transcription factor binding site.

The presence of a chromatin fragment containing a transcription factor and associated TFBS can be used for clinical purposes including for the detection, monitoring, prognosis or treatment selection of or for a disease as described herein. Therefore, in one aspect of the invention there is provided a method for determining the disease status of a subject, for example for the detection, monitoring, prognosis or treatment selection of or for the disease, which comprises the steps of:
(i) contacting a blood sample obtained from the subject with a zinc chelating agent to produce a plasma sample;
(ii) analysing the plasma sample for the presence or level of free DNA fragments containing a DNA sequence including a transcription factor binding site sequence or a flanking sequence to a zinc finger transcription factor binding site; and
(iii) using the presence and/or level and/or sequence of the DNA fragments in the sample as an indicator of the disease status of the subject.

The presence and/or sequence of free DNA fragments among nucleosome or other protein bound DNA fragments in a plasma or other sample may be determined by a number of means including the use of complimentary DNA sequences to bind the DNA fragments in the sample. This may be achieved, for example, by the use of DNA chips which facilitates the probing of the sample for multiple sequences simultaneously. Another embodiment of the invention involves the use of exogenous zinc finger transcription factor as the specific DNA binding agent. In this method the zinc chelating agent is removed to facilitate binding of zinc finger transcription factor to DNA. This may be performed by buffer exchange, for example by dialysis or by using size exclusion chromatography, for example using a sephadex size exclusion chromatography column. DNA fragments containing the TFBS of the zinc finger transcription factor may be isolated, for example by using solid phase bound transcription factor as a binding agent for free DNA containing the TFBS. The isolated DNA may be analysed for sequence and/or DNA fragment length. Recombinant transcription factor proteins may be used for the purposes of the invention. The recombinant zinc finger transcription factor proteins may be linked to a solid phase support or may contain a linker moiety and the transcription factor may be used in liquid form and isolated through the linking system. Many such linking samples are known in the art, for example the zinc finger transcription factor may be biotinylated and isolated using solid phase streptavidin. Therefore, in one embodiment of the invention there is provided a method for identifying the presence of a circulating chromatin fragment containing a zinc finger transcription factor and/or the sequence(s) of DNA fragments bound to the zinc finger transcription factor in a subject which comprises the steps of:
(i) contacting a blood sample obtained from the subject with a zinc chelating agent to produce a plasma sample;
(ii) removing the zinc chelating agent from the sample;
(iii) contacting the sample with an exogenous zinc finger transcription factor; and
(iv) analysing DNA fragments bound by the exogenous transcription factor.

Alternatively or additionally, the zinc chelating agent may simply be inactivated in the sample. In one embodiment of the invention, the zinc chelating agent is inactivated by the addition of excess ions, preferably zinc ions, before contact with exogenous transcription factor. Therefore, in one embodiment of the invention there is provided a method for identifying the presence of a circulating chromatin fragment containing a zinc finger transcription factor and/or the sequence(s) of DNA fragments bound to the zinc finger transcription factor in a subject which comprises the steps of:
(i) contacting a blood sample obtained from the subject with a zinc chelating agent to produce a plasma sample;
(ii) inactivating the zinc chelating agent in the sample by adding excess zinc or other ions;
(iii) contacting the sample with an exogenous zinc finger transcription factor; and
(iv) analysing DNA fragments bound by the exogenous transcription factor.

The presence of a chromatin fragment containing a transcription factor and associated TFBS can be used for clinical purposes including for the detection, monitoring, prognosis or treatment selection of or for a disease as described herein. Therefore, in one aspect of the invention there is provided a method for determining the disease status of a subject, for example for the detection, monitoring, prognosis or treatment selection of or for the disease, which comprises the steps of:
(i) contacting a blood sample obtained from the subject with a zinc chelating agent to produce a plasma sample;
(ii) removing or inactivating the zinc chelating agent in the sample;
(iii) contacting the sample with an exogenous zinc finger transcription factor;
(iv) analysing DNA fragments bound by the exogenous transcription factor, and
(v) using the presence and/or level and/or sequence of the DNA fragments in the sample as an indicator of the disease status of the subject.

### Removal of Cell Free Nucleosomes

The sample preparation may optionally also involve a pre-purification step to remove most of the nucleosomes and nucleosome bound DNA from the sample prior to analysis. This reduces background signal, improves the efficiency of isolation and amplification of the transcription factor bound DNA fragments of interest and may improve the analytical and clinical sensitivity of the methods of the invention. Therefore, in one embodiment, the method additionally comprises removing cell free nucleosomes from the body fluid sample. The chromatin fragments comprising a nucleosome may be removed from the sample (optionally to be analyzed separately) prior to the employment of the methods of the invention described herein. The purpose of this preparative step is to remove the bulk of the DNA fragments from the sample to lower any background signal they may create in the analysis. This may be done for example, without limitation, by contacting the sample with a binding agent which binds to nucleosomes, such as a solid phase anti-nucleosome binder including, for example an antibody or a nucleosome binding protein such as the proteins described in WO2021038010. The antibody may selectively bind to a histone protein, for example a core histone protein such as H2A, H2B, H3 or H4, or a linker histone protein such as H1. References to histone proteins, includes histone post translational modifications and histone variants or isoforms. The nucleosome binding protein may be selected from: a chromatin binding protein which binds to linker DNA or a protein that binds to nucleosome associated linker DNA. For example, the chromatin binding protein which binds to linker DNA may be selected from: a Chromodomain Helicase DNA Binding (CHD) protein; a DNA (cytosine-5)-methyltransferase (DNMT) protein; a High mobility group box protein (HMGB) protein; a Poly [ADP-ribose] polymerase (PARP) protein; or a Methyl-CpG-binding domain (MBD) protein, such as MBD1, MBD2, MBD3, MBD4 or Methyl CpG binding protein 2 (MECP2). The protein which binds to nucleosome associated linker DNA may be selected from histone H1, macroH2A (mH2A), or a fragment or engineered analogue thereof.

All or most of nucleosomal material present in the sample may be adsorbed (e.g. onto the solid phase) and hence removed from the sample. Therefore, in one embodiment, the method comprises contacting the body fluid sample with a binding agent which binds to nucleosomes or a component thereof, and removing the sample bound to the binding agent prior to contacting the sample with a transcription factor binding agent.

It has been reported that a large part of, or most, short cfDNA fragments of less than 100 bp in length in plasma do not derive from chromatin fragments including regulatory proteins, but derive from nucleosome associated DNA which is nicked or broken in one or both DNA strands. In this case the short cfDNA fragments may represent, for example, a 150 bp DNA fragment associated with a nucleosome which is nicked in one or more places to generate two or more smaller cfDNA fragments (for example two fragments of 75 bp) rather than a single 150 bp cfDNA fragment (Sanchez et al, 2018). Therefore, removal of nucleosomes from the sample prior to exposure of the sample to a transcription factor binding agent, has the additional advantage of removal of short cfDNA fragments of less than 100 bp that originate from nucleosome associated nicked DNA. This further reduces the background of nucleosome associated cfDNA in the sample, for example, compared to size separation of extracted cfDNA fragments by gel separation methods.

We have demonstrated quantitative removal of chromatin fragments containing nucleosomes from human plasma samples using an anti-H3 antibody.

In a preferred embodiment, magnetic beads are used as a solid phase support, but any suitable material may be used. Similarly, any of the methods for nucleosome binding methods described by in WO2016067029, WO2017068371 and WO2021038010 as a method of removing nucleosomes may be used. Therefore, in one embodiment, the sample used in methods of the invention does not comprise nucleosomes. In a further embodiment, the cell free chromatin fragment detected by the methods of the present invention consists of a transcription factor and a DNA fragment.

In one embodiment of the invention, there is provided a method of detecting a disease in a human or animal subject which comprises the steps of:
(i) removing a cell free nucleosome from a body fluid sample obtained from the human or animal subject;
(ii) contacting the sample with a binding agent which binds to a transcription factor;
(iii) isolating the DNA associated with the transcription factor;
(iv) amplifying the isolated DNA by a PCR method;
(v) determining the sequence of the amplified DNA; and
(vi) using the presence of the transcription factor and the sequence of the associated DNA as a combined biomarker for determining the presence and/or the nature of a disease in the subject.

In some embodiments of the invention, the presence or sequence of the DNA fragment associated with a cell free transcription factor or chromatin fragment may be determined without isolation of the DNA. This may be done by a variety of methods including, without limitation, amplification methods that do not require DNA isolation.

The term “binding agent” as used herein refers to ligands or binders, such as naturally occurring or chemically synthesized compounds, capable of specific binding to a biomarker (i.e. to a specific transcription factor). A ligand or binder according to the invention may comprise a peptide, an antibody or a fragment thereof, or a synthetic ligand such as a plastic antibody, or an aptamer or oligonucleotide or a molecular imprinted surface or device, capable of specific binding to the biomarker. The antibody can be a monoclonal antibody or a fragment thereof capable of specific binding to the target. A ligand or binder according to the invention may be labelled with a detectable marker, such as a luminescent, fluorescent, enzyme or radioactive marker; alternatively or additionally a ligand according to the invention may be labelled with an affinity tag, e.g. a biotin, avidin, streptavidin or His (e.g. hexa-His) tag. In one embodiment, the binding agent is selected from: an antibody, an antibody fragment or an aptamer. In a further embodiment, the binding agent used is an antibody. The terms “antibody”, “binding agent” or “binder” are used interchangeably herein.

In one embodiment, the sample is a biological fluid (which is used interchangeably with the term “body fluid” herein). Any body fluid sample type may be used for the invention including without limitation blood, plasma, menstrual blood, endometrial fluid, feces, urine, saliva, mucous, semen and breath, e.g. as condensed breath, or an extract or purification therefrom, or dilution thereof. Biological samples also include specimens from a live subject, or taken post-mortem. The samples can be prepared, for example where appropriate diluted or concentrated, and stored in the usual manner. In a preferred embodiment, the biological fluid sample is selected from: blood or serum or plasma. It will be clear to those skilled in the art that the detection of chromatin fragments in a body fluid has the advantage of being a minimally invasive method that does not require biopsy.

In one embodiment, the subject is a mammalian subject. In a further embodiment, the subject is selected from a human or animal (such as a companion animal or a mouse) subject. In a yet further embodiment, the subject is a human subject. In one embodiment, the human subject is a non-embryonic subject (i.e. a human at any stage of development, other than an embryo). In a further embodiment, the human subject is an adult subject, i.e. greater than 16 years or age, such as greater than 18, 21 or 25 years of age. In an alternative embodiment, the subject is an animal subject. In a further embodiment, the animal subject is selected from a rodent (e.g. mouse, rat, hamster, gerbil or chipmunk), feline (i.e. a cat), canine (i.e. a dog), equine (i.e. a horse), porcine (i.e. a pig) or bovine (i.e. a cow) subject.

It will be understood that the uses and methods of the invention may be performed in vitro or ex vivo.

According to a further aspect of the invention there is provided a method for detecting or diagnosing cancer in an animal or a human subject which comprises the steps of:
(i) detecting or measuring DNA associated with a cell free chromatin fragment comprising a transcription factor in a body fluid sample obtained from the subject; and
(ii) using the associated DNA level and/or DNA sequence detected in step (i) to identify the disease status of the subject.

According to a further aspect of the invention there is provided a method for detecting or diagnosing an inflammatory disease in an animal or a human subject which comprises the steps of:
(i) detecting or measuring DNA associated with a cell free chromatin fragment comprising a transcription factor in a body fluid sample obtained from the subject; and
(ii) using the associated DNA level and/or DNA sequence detected in step (i) to identify the inflammatory disease status of the subject.

In one embodiment of the invention, the presence of a cell free chromatin fragment comprising the transcription factor and DNA fragment in a sample is used to determine the optimal treatment regime for a subject in need of such treatment.

According to a further aspect of the invention there is provided a method for assessment of an animal or a human subject for suitability for a medical treatment which comprises the steps of:
(i) detecting, measuring or sequencing DNA associated with a cell free chromatin fragment comprising a transcription factor in a body fluid sample obtained from the subject; and
(ii) using the associated DNA level and/or DNA sequence detected in step (i) as a parameter for selection of a suitable treatment for the subject.

According to a further aspect of the invention there is provided a method for monitoring a treatment of an animal or a human subject which comprises the steps of:
(i) detecting, measuring or sequencing DNA associated with a cell free chromatin fragment comprising a transcription factor in a body fluid sample obtained from the subject;
(ii) repeating the detection, measurement or sequencing of DNA associated with a cell free chromatin fragment comprising the transcription factor in a body fluid of the subject on one or more occasions; and
(iii) using any changes in the associated DNA level and/or DNA sequence detected in step (i) compared to step (ii) as a parameter for any changes in the condition of the subject.

A change in the level of the measured DNA level and/or DNA sequence associated with a cell free chromatin fragment containing a transcription factor detected in the test sample relative to the level or sequence detected in a previous test sample taken earlier from the same test subject may be indicative of a beneficial effect, e.g. stabilization or improvement, of said therapy on the disorder or suspected disorder. Furthermore, once treatment has been completed, the method of the invention may be periodically repeated in order to monitor for the recurrence of a disease.

It will be understood that these aspects of the invention may be used in combination with the methods disclosed herein, e.g. step (i) comprises contacting the body fluid sample with a binding agent which binds to a transcription factor, and then detecting or measuring the DNA associated with said transcription factor.

In one embodiment, the cell free chromatin fragment comprising the transcription factor and DNA fragment (i.e. the DNA associated with the cell free chromatin fragment comprising the transcription factor) is detected or measured as one of a panel of measurements. For example, in combination with the other cell free chromatin transcription factor markers, or with any other biomarkers.

According to a further aspect of the invention there is provided a method for detecting, measuring or sequencing a cell free chromatin fragment comprising a transcription factor and a DNA fragment, either alone or as part of a panel of measurements, for the purposes of determining or assessing an animal or a human subject for suitability for a medical treatment, or for monitoring a treatment of an animal or a human subject, for use in subjects with an actual or suspected cancer or benign tumor.

It will be understood that measurements or assays performed by methods of the invention may include the use of a reference material as a calibrant or positive control to provide a standard against which the output of the assay can be compared or calibrated and/or to confirm or monitor the correct functioning of the chemistry of the assay. Suitable reference materials may include biologically sourced chromatin fragments containing transcription factors or recombinant chromatin fragments including without limitation recombinant transcription factor-DNA complexes.

The terms “detecting” and “diagnosing” as used herein encompass identification, confirmation, and/or characterization of a disease state. Methods of detecting, monitoring and of diagnosis according to the invention are useful to confirm the existence of a disease, to monitor development of the disease by assessing onset and progression, or to assess amelioration or regression of the disease. Methods of detecting, monitoring and of diagnosis are also useful in methods for assessment of clinical screening, prognosis, choice of therapy, evaluation of therapeutic benefit, i.e. for drug screening and drug development.

It will be understood that detecting and measuring includes sequencing. The term “sequencing” as used herein encompasses the determination of the nucleoside base sequence (usually adenine, guanine, thymine and cytosine base sequence) of all or a part of a DNA fragment.

Efficient diagnosis and monitoring methods provide very powerful “patient solutions” with the potential for improved prognosis, by establishing the correct diagnosis, allowing rapid identification of the most appropriate treatment (thus lessening unnecessary exposure to harmful drug side effects), and reducing relapse rates.

It will be understood that identifying and/or quantifying can be performed by any method suitable to identify the presence and/or amount of a specific protein or DNA fragment sequence in a biological sample from a patient or a purification or extract of a biological sample or a dilution thereof. In methods of the invention, quantifying may be performed by sequencing or by measuring the concentration of the biomarker in the sample or samples. Biological samples that may be tested in a method of the invention include those as defined hereinbefore. The samples can be prepared, for example where appropriate diluted or concentrated, and stored in the usual manner.

Identification and/or quantification of biomarkers may be performed by detection of the biomarker or of a fragment thereof, e.g. a fragment with C-terminal truncation, or with N-terminal truncation. Fragments are suitably greater than 4 amino acids in length, for example 5, 6, 7, 8, 9, 10, 11, 12, 13, 14, 15, 16, 17, 18, 19, or 20 amino acids in length.

The biomarker may be directly detected, e.g. by SELDI or MALDI-TOF. Alternatively, the biomarker may be detected directly or indirectly via interaction with a ligand or ligands such as an antibody or a biomarker-binding fragment thereof, or other peptide, or ligand, e.g. aptamer, or oligonucleotide, capable of specifically binding the biomarker. The ligand or binder may possess a detectable label, such as a luminescent, fluorescent or radioactive label, and/or an affinity tag.

For example, detecting and/or quantifying can be performed by one or more method(s) selected from the group consisting of: SELDI (-TOF), MALDI (-TOF), a 1-D gel-based analysis, a 2-D gel-based analysis, Mass spec (MS), reverse phase (RP) LC, size permeation (gel filtration), ion exchange, affinity, HPLC, UPLC and other LC or LC MS-based techniques. Appropriate LC MS techniques include ICAT® (Applied Biosystems, CA, USA), or iTRAQ® (Applied Biosystems, CA, USA). Liquid chromatography (e.g. high pressure liquid chromatography (HPLC) or low pressure liquid chromatography (LPLC)), thin-layer chromatography, NMR (nuclear magnetic resonance) spectroscopy could also be used.

It will be understood that detecting and/or measuring DNA may comprise, for example, hybridization or sequencing as described herein.

Methods of diagnosing or monitoring according to the invention may comprise analyzing a sample by SELDI TOF or MALDI TOF to detect the presence or level of the biomarker. These methods are also suitable for clinical screening, prognosis, monitoring the results of therapy, identifying patients most likely to respond to a particular therapeutic treatment, for drug screening and development, and identification of new targets for drug treatment.

Identifying and/or quantifying the analyte biomarkers may be performed using an immunological method, involving an antibody, or a fragment thereof capable of specific binding to the biomarker.

According to a further aspect of the invention, there is provided a method for identifying a cell free chromatin fragment comprising a transcription factor and a DNA fragment as a combination biomarker for detecting or diagnosing a disease in an animal or human subject which comprises the steps of:
(i) detecting and/or measuring and/or sequencing a cell free chromatin fragment comprising a transcription factor and DNA fragment combination biomarker in a body fluid sample of a diseased subject;
(ii) detecting and/or measuring and/or sequencing a cell free chromatin fragment comprising the transcription factor and DNA fragment combination biomarker in a body fluid sample of a healthy subject or a control subject; and
(iii) using the difference between the levels and/or DNA sequences detected in diseased and healthy or control subjects to identify whether a cell free chromatin fragment comprising the transcription factor and DNA fragment combination biomarker is useful as a biomarker for the disease status.

It will be appreciated that this aspect of the invention may be combined with the methods described herein, i.e. steps (i) and/or (ii) may be performed using a method as defined herein.

According to a further aspect of the invention, there is provided a biomarker or combined biomarker identified by the method described herein.

Diagnostic or monitoring kits are provided herein for performing methods of the invention. Such kits for detection and/or quantification of the biomarker or combined biomarker will suitably comprise a ligand or binder for the transcription factor and optionally reagents for the amplification and/or sequencing of DNA associated with said transcription factor and optionally a ligand or binder for nucleosomes, optionally together with instructions for use of the kit. Biomarker monitoring methods, biosensors and kits are also vital as patient monitoring tools, to enable the physician to determine whether relapse is due to worsening of the disorder. If pharmacological treatment is assessed to be inadequate, then therapy can be reinstated or increased; a change in therapy can be given if appropriate. As the biomarkers are sensitive to the state of the disorder, they provide an indication of the impact of drug therapy.

According to a further aspect of the invention there is provided a kit for the detection of a cell free chromatin fragment comprising a transcription factor and a DNA fragment as a combination biomarker which comprises a ligand or binder for the transcription factor, optionally reagents for the amplification and/or sequencing of DNA associated with said transcription factor, and optionally a ligand or binder for nucleosomes, optionally together with instructions for use of the kit in accordance with the methods described herein.

A further aspect of the invention is a kit for detecting the presence of a disease state, comprising a biosensor capable of detecting and/or quantifying one or more of the biomarkers as defined herein.

According to a further aspect, there is provided the use of a kit as defined herein for the diagnosis of cancer. According to a further aspect, there is provided the use of a kit as defined herein for the diagnosis of an inflammatory disease. According to a further aspect, there is provided the use of a kit as defined herein for the diagnosis of a prenatal disease.

According to a further aspect, there is provided a method of treating a disease in a subject in need thereof, wherein said method comprises the following steps:
(a) contacting a body fluid sample obtained from the human or animal subject with a binding agent which binds to a transcription factor;
(b) detecting, measuring or sequencing a DNA fragment associated with the transcription factor; and
(c) using the presence, amount or sequence of the DNA fragment as an indicator of the presence of the disease in the subject; and
(d) administering a treatment if the subject is determined to have the disease in step (c).

In one embodiment, the disease is cancer. In an alternative embodiment, the disease is an inflammatory disease. According to a further aspect, there is provided the use of a kit as defined herein for the diagnosis of a prenatal disease in a fetus of a pregnant subject.

In one embodiment, the treatment administered is selected from: surgery, radiotherapy, chemotherapy, immunotherapy, hormone therapy and biological therapy.

According to a further aspect of the invention, there is provided a method of treating cancer in a subject in need thereof, wherein said method comprises the following steps:
(a) detecting or diagnosing cancer in the subject according to the method described herein; followed by
(b) administering an anti-cancer therapy, surgery or medicament to said individual.

In one embodiment, the subject is a human or an animal subject.

We now illustrate the invention with the following examples.

### EXAMPLES

### Example 1

An antibody directed to bind specifically to the transcription factor TTF-1 (also called NKX2-1) is coated onto magnetic beads for biomagnetic separation (e.g. commercially available Dynabeads). TTF-1 is a homeobox helix-turn-helix transcription factor.

Anti-TTF-1 antibody coated magnetic beads are added to EDTA plasma samples collected from human subjects diagnosed with stage IV lung cancer, stage IV thyroid cancer and from healthy subjects. Following incubation (with gentle rotation to maintain the suspension of magnetic particles), the magnetic particles are removed from the plasma samples and washed with assay buffer. TTF-1 associated DNA fragments are isolated from the magnetic solid phase using the Qiagen QiaAMP Circulating Nucleic Acids kit. Adapter oligonucleotides are ligated to the isolated DNA fragments to produce a single stranded DNA library of DNA sequences associated with TTF-1 for each plasma sample by the library method described in Snyder et al, 2016, which is herein incorporated by reference.

The fragment libraries produced for each subject are amplified by real-time quantitative PCR. The amplified libraries are sequenced using next generation sequencing methods and the amounts of DNA in each library and the sequences associated are compared. The coverage of the TTF-1 TFBS loci by small cfDNA fragments in the 35-80 bp range will be low in healthy samples because the amounts of TTF-1 associated DNA in the healthy samples will be low or undetectable. In contrast, the coverage of the TTF-1 TFBS loci by small cfDNA fragments in the 35-80 bp range will be high in the cancer samples because the amounts of TTF-1 associated DNA in the samples in patients with stage IV lung cancer or stage IV thyroid cancer is higher. The sequences of the associated TTF-1 DNA determined in the thyroid cancer samples will correlate to the known sequences of TTF-1 regulated gene promoters in thyroid cells. Similarly, the sequences of the associated TTF-1 DNA determined in the lung cancer samples will correlate to the known sequences of TTF-1 regulated gene promoters in thyroid cells. On this basis most or all of the healthy samples, thyroid cancer samples and lung cancer samples will be identifiable from the data produced by the experiment.

### Example 2

The experiment described in EXAMPLE 1 is repeated but prior to incubation with magnetic particles coated with an anti-TTF1 antibody, magnetic beads coated with an anti-nucleosome antibody are added to the plasma samples to preclear the samples of nucleosomes and nucleosome bound DNA fragments. Following incubation (with gentle rotation to maintain the suspension of magnetic particles), the magnetic particles are removed from the plasma sample. The experiment is then completed as described in EXAMPLE 1 using the remaining sample, with similar results except that the background levels of DNA found in healthy samples will be even lower than described for EXAMPLE 1.

### Example 3

Anti-TTF-1 antibody coated magnetic beads are added to EDTA plasma samples collected from human subjects with stage IV lung cancer, stage IV thyroid cancer and from healthy subjects. Following incubation (with gentle rotation to maintain the suspension of magnetic particles), the magnetic particles are removed from the plasma samples and washed with assay buffer. TTF-1 associated DNA fragments are extracted from the magnetic solid phase using the Qiagen QiaAMP Circulating Nucleic Acids kit. Specific sequence primers are designed using typical software known in the art for primer design, to amplify DNA fragments of specific sequences associated with the TTF-1 binding sites in the SPB, thyroid stimulating hormone receptor and thyroperoxidase gene promoter regions of the human genome plus flanking DNA. The primers are used to amplify the DNA fragments by real-time quantitative PCR. The amount of DNA present is measured for each sequence in each plasma sample. The results for samples taken from healthy subjects will be low or undetectable. Most samples taken from lung cancer patients will contain detectable amounts of SPB gene promoter sequence DNA fragments. Most samples taken from thyroid cancer patients will contain detectable amounts of thyroid stimulating hormone receptor and/or thyroperoxidase gene promoter sequence DNA fragments. On this basis most or all of the healthy samples, thyroid cancer samples and lung cancer samples will be identifiable from the data produced by the experiment.

### Example 4

The experiment described in EXAMPLE 3 is repeated but prior to incubation with magnetic particles coated with an anti-TTF-1 antibody, magnetic beads coated with an anti-nucleosome antibody are added to the plasma samples to preclear the samples of nucleosomes and nucleosome bound DNA fragments. Following incubation (with gentle rotation to maintain the suspension of magnetic particles), the magnetic particles are removed from the plasma sample. The experiment is then completed as described in EXAMPLE 3 using the remaining sample, with similar results except that the background levels of DNA found in healthy samples will be even lower than described for EXAMPLE 3.

### Example 5

Similar experiments to those described in the above examples are repeated for the helix-turn-helix transcription factor NKX3.1 by testing in plasma samples collected from healthy men and from men diagnosed with stage IV prostate cancer. The results for samples taken from healthy subjects will be low or undetectable. Most samples taken from prostate cancer patients will contain detectable amounts of NKX3.1 gene promoter sequence DNA fragments in the 35-80 bp size range. On this basis most or all of the healthy samples and prostate cancer samples will be identifiable from the data produced by the experiment.

### Example 6

Similar experiments to those described in the above examples are repeated for the zinc finger transcription factor WT1 by testing in serum samples collected from healthy women and from women diagnosed with stage IV ovarian cancer. The results for samples taken from healthy subjects will be low or undetectable coverage of WT1 TFBS loci by WT1 associated cfDNA fragments of 35-80 bp size range in healthy subjects. Most samples taken from ovarian cancer patients will show higher coverage of WT1 TFBS loci by WT1 associated cfDNA fragments of 35-80 bp size range as they contain detectable amounts of WT1 gene promoter sequence 35-80 bp cfDNA fragments. On this basis most or all of the healthy samples and ovarian cancer samples will be identifiable from the data produced by the experiment.

### Example 7

We coated Dynabeads M280 Tosyl activated magnetic beads with an antibody directed to bind to a histone H3 epitope located at amino acid position 30-33. This antibody was selected from a number of antibodies tested as it was observed to bind to both nucleosomes containing full histone tails and to nucleosomes with clipped histone tails.

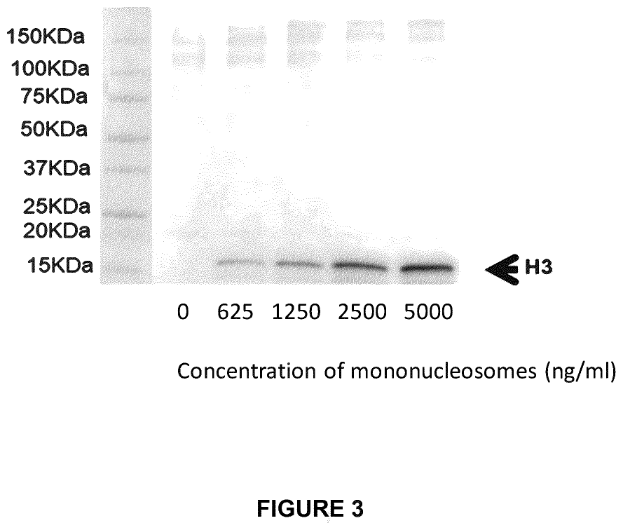

We added anti-H3 antibody coated magnetic beads (1 mg) to solutions containing a range of concentrations of recombinant mononucleosomes (0.5 ml) purchased from Active Motif. The beads were incubated with the nucleosomes at room temperature for 1 hour with gentle rolling of the tubes to maintain the beads in suspension. The beads were isolated magnetically and washed. Nucleosomes adsorbed to the beads were then removed by elution and analyzed by Western blot. The results demonstrate that the nucleosomes were adsorbed from solution by the magnetic beads in a dose dependent fashion as shown in FIG. 3.

### Example 8

Anti-H3 antibody coated magnetic beads were prepared and used as described in Example 7. We added anti-H3 antibody coated magnetic beads, as well as uncoated beads, to 8 human EDTA plasma samples as well as to solutions containing a range of concentrations of recombinant mononucleosomes. The range of recombinant mononucleosomes concentrations was selected to include levels typically observed in human clinical samples.

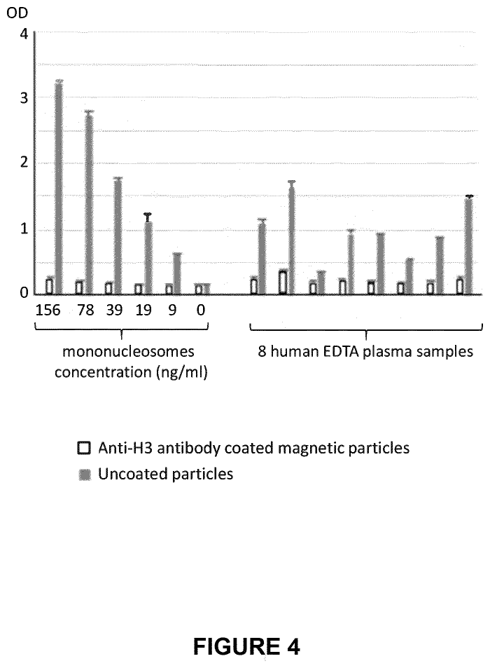

We tested for the presence of nucleosomes remaining in solution following incubation with magnetic beads using an ELISA for nucleosomes with an optical density (OD) readout. The results shown in FIG. 4, demonstrate that the level of recombinant mononucleosomes remaining in solution, following adsorption with anti-H3 antibody coated magnetic beads, was undetectable (had a similar OD to the control solution which contained no nucleosomes) whilst the levels in the solutions incubated with uncoated magnetic beads were unaffected leading to a normal ELISA dose response curve. Similarly, the level of nucleosomes remaining in solution in 8 human plasma samples tested, following adsorption with anti-H3 antibody coated magnetic beads, was also low or undetectable but was not affected by incubation with uncoated magnetic beads. These results demonstrate quantitative removal of nucleosomes from human plasma samples.

### Example 9

Luminex beads of different colours are coated with antibodies directed to bind to the transcription factors TTF-1, NKX3.1, GATA-3, CDX-2 and GRHL2 according to the manufacturer's protocol. Plasma samples taken from healthy subjects and from subjects diagnosed with a variety of cancers are contacted with mixtures of all the beads. The amount or coverage of cfDNA in the 35-80 bp range covering the respective transcription factor TFBS bound to each bead-bound transcription factor is measured by a PCR method or by next generation sequencing. The results will show that the NKX3.1 and GRHL2 TFBS coverage by 35-80 bp cfDNA bound to beads coated with antibodies directed to bind NKX3.1 and GRHL2 is elevated in samples taken from prostate cancer patients whilst transcription factor binding to other beads (coated with anti-TTF-1, GATA-3 or CDX-2 antibody) is low. Similarly, the quantity of short 35-80 bp cfDNA fragments bound to beads coated with antibodies directed to bind TTF-1 and GRHL2 will be elevated in samples taken from lung cancer patients whilst transcription factor binding to other beads (coated with anti-NKX3.1, GATA-3 or CDX-2 antibody) is low. Similarly, the quantity of short 35-80 bp cfDNA fragments bound to beads coated with antibodies directed to bind GATA-3 and GRHL2 will be elevated in samples taken from breast cancer patients whilst the binding to other beads (coated with anti-TTF-1, NKX3.1 or CDX-2 antibody) is low. In contrast the binding of short 35-80 bp cfDNA fragments to all beads will be low in samples taken from healthy subjects.

### Example 10

Magnetic beads are coated with antibodies directed to bind to RNA polymerase II according to the manufacturer's protocol. Plasma samples taken from healthy subjects and from subjects diagnosed with a variety of cancers are contacted with the beads. The beads are washed to remove unbound chromatin fragments.

The DNA bound to the beads is extracted, linked to adapter oligonucleotides and the library is sequenced to find the set of active genes present in the subjects' samples. The results will show that the active genes present in samples taken from healthy subjects are representative of genes active in hematopoietic cells. The same sequences are also present in the samples taken from patients with cancer, but these samples are found to additionally contain RNA polymerase II associated DNA sequences representing genes not active in hematopoietic cells but active in the cells of the disease tissue including genes that are typically active in the (healthy or diseased) cells of the tissue concerned and/or that are upregulated in cancer cells.

### Example 11

Magnetic beads are coated with antibodies directed to bind to RNA polymerase II according to the manufacturer's protocol. Plasma samples taken from healthy subjects and from subjects diagnosed with a variety of cancers are contacted with the beads. The beads are washed to remove unbound chromatin fragments.

The DNA bound to the beads is analysed for the presence of a specific DNA sequence using PCR primers to amplify the sequence. The sequence to be analysed is selected to be associated specifically with colorectal cancer. The results will show that the sequence is present in samples taken from subjects with colorectal cancer, but is not present in samples taken from healthy subjects or from subjects with other cancers.

### Example 12

EDTA plasma samples were collected from 6 women diagnosed with ovarian cancer, 2 women diagnosed with ER-negative breast cancer and 8 women diagnosed with ER-positive breast cancer, of whom 4 women were diagnosed with an ER score of 7 and 4 women were diagnosed with an ER score of 8. The EDTA plasma samples were assayed for ERα using a commercial ERα ELISA kit. The quantitative detection range of the ELISA kit used was 3-200 pg/ml with a lower limit of detection for ERα of 0.8 pg/ml. The mean measured levels of ERα were low for ER-negative subjects and higher for subjects diagnosed with ovarian cancer or ER-positive breast cancer. Moreover, the mean level measured for subjects diagnosed with ER-positive breast cancer was higher for those women with a higher ER score (FIG. 5). We conclude that the presence of ERα in EDTA plasma samples prepared from whole blood samples taken from women is useful as a biomarker for gynecological diseases including gynecological cancers.

### Example 13

The progesterone receptor status of breast cancer as PR-positive or PR-negative is similarly important in the diagnosis and treatment of gynecological cancers. We further conclude that the measurement of progesterone receptor (PR) levels in EDTA plasma samples prepared from whole blood samples taken from women is similarly useful as a biomarker for gynecological diseases including gynecological cancers.

### Example 14

The androgen receptor status of prostate cancer is similarly important in the diagnosis and treatment of prostate cancer. We further conclude that the measurement of androgen receptor (AR) levels in EDTA plasma samples prepared from whole blood samples taken from men is useful as a biomarker for prostate disease including prostate cancer.

### Example 15

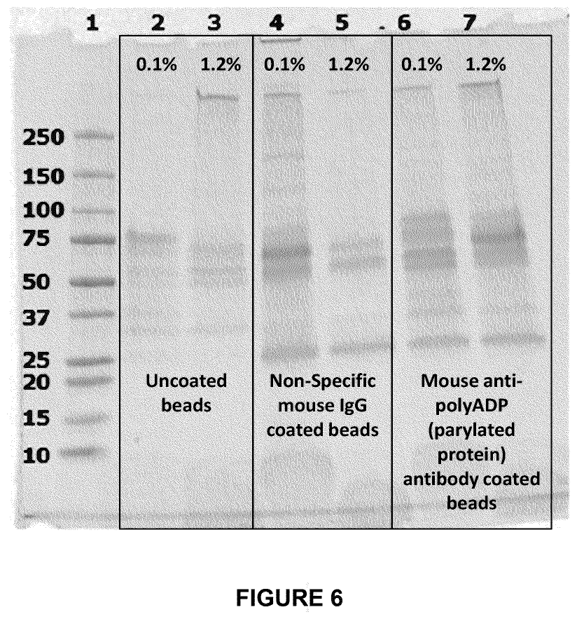

The background level of proteins adsorbed from a plasma sample non-specifically to (non-specific) mouse IgG coated magnetic particles was assessed by Western blot using Coomassie blue stain for development. The background was assessed after 5 washes of the particles with a typical immunochemical wash buffer containing 0.1% Tween 20 detergent or with a wash buffer containing a high level of 1.2% of a mixture of detergents comprising 1% octylphenoxypolyethoxyethanol detergent, 0.1% sodium deoxycholate and 0.1% sodium dodecyl sulfate. The results (FIG. 6) show that the background staining was much reduced by using strong detergents.

The same experiment was applied to proteins adsorbed specifically to a mouse anti-polyADP antibody (that binds parylated proteins of any size). In this case the staining was less affected showing that washing removes non-specifically bound proteins but does not affect (or has less affect) on specifically bound proteins attached to an antibody.

### Example 16

We coated a monoclonal antibody directed to bind specifically to the transcription factor CTCF onto magnetic beads (MyOne TosylActivated Dynabeads™) using standard methods. Briefly 0.86 mg monoclonal antibody was incubated with 29 mg magnetic beads (30 μg antibody/mg of bead) in 2.9 ml 0.1M Borate Buffer pH9.5 containing 1M Ammonium Sulfate for 18 hours at 37° C. in a rolling bottle to maintain suspension of the beads. The beads were sedimented and the supernatant was decanted. The beads were resuspended and incubated for 1 hour at 37° C. in 2.9 mL of a blocking buffer of phosphate buffered saline pH7.4 (PBS) containing 0.1% Tween 20 and 1% bovine serum albumin (BSA). The beads were then sedimented, washed twice with 3 mL PBS containing 0.1% Tween 20 and 1% BSA and stored in 2.9 mL PBS containing 0.1% Tween 20, 1% BSA and a preservative. Non-specific mouse IgG was similarly coated to magnetic beads as a non-specific control reagent.

Chromatin immunoprecipitation (ChIP) of CTCF-DNA fragments was performed in 4 pooled cross-linked EDTA plasma samples obtained from cancer patients (1.6 mL collected in Streck Cell-Free DNA BCTs). Each pooled sample was diluted with 0.4 mL of a commercially available radioimmunoprecipitation assay buffer and 1 mg of anti-CTCF coated magnetic particles was added. The mixture was incubated 1 hour at room temperature with rolling to maintain suspension of the beads. The beads were then sedimented and washed 5 times with a strong detergent wash solution containing a mixture of 1% Triton X-100 detergent, 0.1% sodium deoxycholate and 0.1% sodium dodecyl sulfate and stored in 0.1 mL of buffer. In parallel, a control experiment was performed by incubating 1.6 mL of each pooled plasma sample with non-specific mouse IgG coated magnetic beads.

Following incubation of the magnetic particles with the pooled plasma samples, the magnetic particle bound protein was suspended in a denaturing 1% sodium dodecyl sulphate (SDS) buffer and the denatured protein was analysed by Western blot using an anti-CTCF antibody and a labelled anti-mouse antibody for detection. In Western blot experiments, the presence of CTCF is indicated by the presence of a band at 130-140 kD (Klenova et al, 1997). The results of the Western blot analysis are shown in FIG. 7. Briefly a protein band at approximately 140 kD corresponding to the presence of CTCF transcription factor was visible for all the 4 samples when exposed to magnetic particles coated with anti-CTCF antibody (Anti CTCF). In contrast, no band was visible for any of the same 4 samples exposed to magnetic particles coated with a non-specific mouse IgG (NS-IgG). This indicates that the ChIP method employed was able to selectively isolate circulating transcription factor CTCF from all 4 pooled samples tested. It also demonstrates the clean background produced by the washing regime employed.

### Example 17

CTCF is a zinc finger transcription factor. Chromatin immunoprecipitation (ChIP) of CTCF-DNA fragments was performed in a cross-linked EDTA plasma sample (2.4 mL collected in a Streck Cell-Free DNA BCT) obtained from a subject diagnosed with breast cancer. ChIP was performed as described above in EXAMPLE 16 except the 2.4 mL sample was diluted with 0.6 mL of a radioimmunoprecipitation assay buffer and 1.5 mg anti-CTCF coated magnetic particles were added. In a parallel control experiment, 2.4 mL of the cross-linked EDTA plasma sample was incubated with magnetic beads coated with non-specific mouse IgG. The magnetic beads were split in 2 fractions. One fraction was used for analysis by Western Blot which confirmed the presence of CTCF protein on the beads using fragmented chromatin from MCF7 breast cancer cells as a positive control.

The second fraction of (test and control) beads was used for DNA extraction and analysis. The cross-linking of the magnetic bead associated chromatin fragments with associated DNA was reversed by heating for 15 min at 95° C. The DNA associated with the magnetic beads was then extracted using a commercially available DNA extraction kit (Qiagen QIAamp DSP circulating NA kit) according to the manufacturer's instructions.

The extracted cfDNA was amplified to produce a single strand library for sequencing using a commercially available kit (Claret Bio SRSLY NGS Library Prep Kit) according to the manufacturer's instructions using 16 amplification cycles. The amplified test and non-specific cfDNA fragment libraries were analysed by electrophoresis using a Bioanalyzer instrument. The results (FIG. 8) show that the amplified cfDNA library obtained from the specific anti-CTCF coated magnetic particles contained small fragments in the 35-80 bp range. Note that the sharp peak in the electropherogram at approximately 140 bp represents the adapter dimer, so adapter linked fragments of 175-220 bp represent cfDNA fragments of 35-80 bp. The major peak of adapter ligated cfDNA fragments was observed at approximately 190 bp which corresponds to cfDNA fragments of approximately 50 bp in length. Although, the amplified cfDNA library contained small fragments in the 35-80 bp range, not all these fragments were bound to CTCF in the sample because small DNA fragments were also obtained for amplified extracts from solid supports coated with non-specific mouse IgG. However, the specific peak obtained with specific anti-CTCF antibody ChIP (1000 fluorescence units [FU]) was higher than the non-specific IgG peak (80 FU). This sample was sent for sequencing.

### Example 18

An amplified cfDNA library was prepared from a cross-linked EDTA plasma sample (collected in Streck cfDNA BCTs) collected from a patient diagnosed with colorectal cancer (CRC), by anti-CTCF immunoprecipitation as described in EXAMPLE 17 above. The amplified cfDNA library isolated using anti-CTCF immunoprecipitation was sequenced by Next Generation Illumina NovaSeq sequencing.

Sequenced reads, each representing a cfDNA fragment, were aligned to the human reference genome GRCh38/hg38 using the Illumina DRAGEN Bioinformatics pipeline (https://emea.illumina.com/products/by-type/informatics-products/dragen-bio-it-platform.html). Any non-aligned reads were discarded. The resulting alignment BAM files were used to create subsets of different fragment sizes (35-80 bp, 135-155 bp and 156-180 bp) using Sequence Alignment/Map SAMtools (Li et al, 2009). Read coverage (the number of fragments found to cover a specific gene locus) was calculated using a bin size of 1 bp (the highest resolution possible). Read coverage was normalized to the total number of reads mapped to the human genome with the RPGC (reads per genome coverage) using the deepTools bamCoverage. The coverage profile plots (FIGS. 9 and 10) were generated for each fragment size using deepTools plotProfile (Ramírez et al, 2016).

Results for the coverage at the loci of 9780 published CTCF binding sites (Kelly et al, 2012) by short 35-80 bp fragments associated with CTCF in comparison to coverage by longer cfDNA fragments consistent with sizes expected for circulating mononucleosome association (135-155 bp and 156-180 bp) is shown in FIG. 9(a). The coverage is shown over a 5000 bp range including 2500 bases upstream and downstream of the CTCF binding site location. We observed a strong peak of coverage by small 35-80 bp cfDNA fragment binding at exactly the genomic positions of the CTCF TFBS loci reported by Kelly et al, 2012. Because the sequenced library was produced directly from cfDNA attached to CTCF protein isolated on anti-CTCF coated magnetic beads with a low background, the cfDNA library contained few nucleosomes and the nucleosome positioning signal was low. This feature produces a clear 35-80 bp signal and eliminates the need for deconvolution of competing signals in mixed samples (for example samples containing mixed cfDNA fragments originating from hematopoietic and cancer tissues). By contrast, the cfDNA library obtained for binding to non-specific mouse IgG showed no peak at CTCF TFBS loci (FIG. 9(b)).

A large number of proteins may bind to, or near to, a TFBS including a transcription factor, or any combination of a variety of cooperatively binding transcription factors, transcription enhancers, repressors or other regulatory proteins. A major advantage of the method of the present invention is that the small cfDNA fragment coverage of CTCF TFBS loci is known to relate only to cfDNA fragments associated with CTCF. In contrast methods in the art, for example the fragmentomics methods of Snyder et al, 2016 and Ulz et al, 2019, map all cfDNA fragments of all sizes extracted from EDTA plasma and infer that protein binding did or did not occur at any particular genomic location. Which protein was involved cannot be known because the first step of all such methods is the extraction of cfDNA which entails the disassociation of all nucleoprotein chromatin fragments (including nucleosomes and transcription factor-DNA complexes) in the sample, and hence destroys any direct information linking any particular cfDNA sequences to any particular transcription factor or other protein.

Peak calling of the cfDNA fragment sequences with reference to the input non-specific control resulted in CTCF as the transcription factor with the most TFBS sequence fragments. Peak calling was performed on the BAM files using MACS2 (Zhang et al, 2008) narrow peaks. The peaks files were used to detect transcription factor binding sites using findMotifGenome tool from Homer Software package (Heinz et al, 2010).

We then repeated the analysis for enrichment of 1041 CTCF TFBS occupied in immortalized cancer cells (Liu et al, 2017). The results shown in FIG. 10(a) show that there was a clear peak of 35-80 bp CTCF associated cfDNA fragment binding to the 1041 cancer specific CTCF TFBS sequences. Unlike fragmentomics, the cfDNA fragments contributing to the analysis originate from CTCF-DNA complexes only and not to other transcription factor-DNA or cofactor-DNA complexes if they do not include CTCF. This demonstrates CTCF occupancy of the cancer specific loci and hence also indicates a tumor cell origin for those cfDNA fragments and the CTCF-DNA complexes from which they derived. There was no peak for longer (nucleosome size) cfDNA fragments. The cfDNA library obtained for binding to non-specific mouse IgG showed no peak (FIG. 10(b)).

The demonstration that CTCF associated cfDNA fragments were bound to cancer specific TFBS loci in a body fluid by ChIP-Seq is indicative of the presence of a cancer disease in the subject investigated and can be used as a biomarker in this manner. We conclude that the methods of the invention are successful for the ChIP-Seq of transcription factors in plasma and as a biomarker for disease.

### Example 19

The androgen receptor (AR) is a zinc finger transcription factor of interest in prostate cancer. We applied the same method described for CTCF in EXAMPLE 17 to AR. We used a mouse anti-AR antibody to immunoprecipitate AR from cross-linked EDTA plasma samples (collected in Streck cfDNA BCTs) from 8 subjects diagnosed as suffering from prostate cancer. We performed Western blot analysis of the protein isolated by ChIP on the solid phase support using AR from LnCAP prostate cancer cell line cells as a positive control. The results in FIG. 11 show that a protein band corresponding to AR at a molecular weight of approximately 100 kD was present in all 8 samples and at high levels in 2 samples (lanes 2 and 3 of FIG. 11). The band at approximately 50 kD corresponds to binding of the labelled anti-mouse IgG antibody to the heavy chain of the mouse anti-AR antibody employed for ChIP. We then extracted DNA from the solid phase supports, ligated the extracted DNA fragments to adapter oligonucleotides and amplified the DNA present. The results (FIG. 12) show that the amplified cfDNA library contained small fragments in the 35-80 bp range (adapter linked fragments of 175-220 bp) for all 8 samples. Although, the amplified cfDNA library contained small fragments in the 35-80 bp range, not all these fragments were bound to AR in the sample because small DNA fragments were also obtained for amplified extracts from solid supports coated with non-specific mouse IgG. The amplified cfDNA libraries obtained for the 2 samples with the highest observed levels of AR by Western were then sequenced by Next Generation Sequencing.

### REFERENCES

Active Motif, Nat. Methods 3: 658 (2006), doi: 10. 1038/NMETH907
Bohinski et al. Molecular and Cellular Biology, 14(9): 5671 (1994)
Corces et al. Science, 362(6413): eaav1898 (2018), doi: 10.1126/science.aav1898.
Crowley et al. Nat. Rev. Clin. Oncol. 10: 472-484 (2013), doi: 10.1038/nrclinonc.2013.110
Darnell, Nat. Rev. Cancer 2: 740-749 (2002), doi: 10.1038/nrc906
Deligezer et al. Clinical Chemistry 54:7 1125-1131 (2008)
Dunbar, Clinica Chimica Acta 363 (1-2): 71-82 (2006), doi.org/10.1016/j.cccn.2005.06.023
Gurel et al. Am J Surg Pathol, 34(8): 1097-105 (2010), doi: 10.1097/PAS.0b013e3181e6cbf3.
Heinz et al. Mol. Cell 38(4): 576-89 (2010), doi: 10.1016/j.molcel.2010.05.004.
Holdenrieder & Stieber, Crit. Rev. Clin. Lab. Sci. 46(1):1-24 (2009), doi: 10.1080/10408360802485875
Hu et al. J. Trans. Med. 17: 124 (2019), doi: 10.1186/s12967-019-1871-x
Jung et al. Clin. Chim. Acta 411(21-22): 1611-24 (2010), doi:10.1016/j.cca.2010.07.032 Kelly et al. Genome Res. 22: 2497-2506 (2012), doi: 10.1101/gr.143008.112.
Klenova et al. Nucleic Acids Res. 25(3): 466-473 (1997), doi.org/10.1093/nar/25.3.466
Lambert et al. Cell 172(4):650-665 (2018), doi:10.1016/j.cell.2018.01.029
Latil et al. Cell Stem Cell 20(2): 191-204.e5 (2017), doi:10.1016/j.stem.2016.10.018.
Lee et al. J. Mol. Med. (Berl). 85(12): 1393-404 (2007), doi: 10.1007/s00109-007-0237-7
Li et al. Bioinformatics 25(16): 2078-2079 (2009), doi: 10.1093/bioinformatics/btp352 Lin et al. PLOS Genet. 3(6):e87 (2007), doi:10.1371/journal.pgen.0030087.eor
Liu et al. Oncotarget 8(69): 114183-114194 (2017), doi: 10.18632/oncotarget.23172
Liu et al. EBioMedicine 41: 345-356 (2019), doi: 10.1016/j.ebiom.2019.02.010
Maenhaut et al. 2015 In: Feingold, Anawalt, Boyce, et al., editors. Endotext. https://www.ncbi.nlm.nih.gov/books/NBK285554/
Mann et al. Curr. Top Dev. Biol. 88: 63-101 (2009), doi: 10.1016/S0070-2153(09)88003-4.
Mansson et al. Mol. Oncol. 15(11): 2868-2876 (2021), doi: 10.1002/1878-0261.13093
Matys et al. Nucleic Acids Res. 34: D108-D110 (2006), doi: 10.1093/nar/gkj143
Merabet and Mann, Trends Genet. 32(6): 334-347 (2016), doi: 10.1016/j.tig.2016.03.004.
Newman et al. Nat. Med. 20(5): 548-54 (2014), doi: 10.1038/nm.3519
Park et al. Oncol. Lett. 3(4): 921-926 (2012), doi: 10.3892/ol.2012.592
Pomerantz et al. Nat. Genet. 47(11): 1346-51 (2015), doi:10.1038/ng.3419.
Poorey et al. Science 342(6156): 369-72 (2013), doi: 10.1126/science. 1242369.
Ramírez et al. Nucleic Acids Res. 44(W1): W160-5 (2016), doi: 10.1093/nar/gkw257
Ralston, Do transcription factors actually bind DNA? DNA footprinting and gel shift assays. Nature Education 1(1): 121 (2008)
Sadeh et al. Nat. Biotechnol. 39: 586-598 (2021), doi.org/10.1038/s41587-020-00775-6
Sanchez et al. NPJ Genom. Med. 3: 31 (2018), doi: 10.1038/s41525-018-0069-0
Skene and Henikoff, eLife 6:e21856 (2017), doi: 10.7554/eLife.21856.002
Snyder et al. Cell 164(1-2): 57-68 (2016), doi: 10.1016/j.cell.2015.11.050
Ulz et al. Nat. Commun. 10(1): 4666 (2019), doi: 10.1038/s41467-019-12714-4
Vad-Nielsen et al. Lung Cancer 147: P244-251 (2020), doi.org/10.1016/j.lungcan.2020.07.023
Vaquerizas et al. Nat. Rev. Genet. 10(4): 252-63 (2009), doi: 10.1038/nrg2538
Wang et al. Genome Res. 22(9): 1680-8 (2012), doi: 10.1101/gr.136101.111
Zhang et al. Genome Biol. 9(9): R137 (2008), doi: 10.1186/gb-2008-9-9-r137
Zhou et al. BMC Genomics 18(1):724 (2017), doi: 10.1186/s12864-017-4115-6

1. A method of detecting a cell free chromatin fragment comprising a transcription factor and a DNA fragment in a body fluid sample obtained from a human or animal subject, which comprises the steps of:
(i) contacting the body fluid sample with a binding agent which binds to the transcription factor;
(ii) detecting or measuring the DNA fragment associated with the transcription factor; and
(iii) using the presence or amount of the DNA fragment as a measure of the amount of cell free chromatin fragments comprising the transcription factor in the sample.

2. The method of claim 1, which comprises isolating the transcription factor bound in step (i) from the remaining body fluid sample, prior to detection of the associated DNA fragment in step (ii).

3. The method of claim 1, wherein step (ii) comprises sequencing the DNA fragment associated with the transcription factor.

4. The method of claim 1, which additionally comprises extracting the DNA fragment associated with the transcription factor.

5. The method of claim 4, which additionally comprises amplification of the extracted DNA fragment, such as by PCR.

6. The method of claim 1, wherein the DNA fragment associated with the transcription factor is detected and/or measured by real-time PCR.

7. The method of claim 1, further comprising removing cell free nucleosomes from the body fluid sample.

8. The method of claim 7, further comprising contacting the body fluid sample with a binding agent which binds to nucleosomes or a component thereof and removing the sample bound to the binding agent prior to step (ii).

9. The method of claim 1, wherein the cell free chromatin fragment consists of the transcription factor and DNA fragment.

10. The method of claim 1, wherein the transcription factor bound by the binding agent in step (i) is washed with a buffer solution containing at least 1% concentration of detergent, prior to detection of the associated DNA fragment in step (ii).

11. A method of detecting a disease in a human or animal subject which comprises the steps of:
(i) contacting a body fluid sample obtained from the human or animal subject with a binding agent which binds to a transcription factor;
(ii) detecting or measuring the DNA associated with the transcription factor; and
(iii) using the presence or amount of DNA as an indicator of the presence of a disease in the subject.

12. The method of claim 11, further comprising using the transcription factor and sequence of the DNA fragment as a combined biomarker for indicating the presence of the disease in the subject.

13. The method of claim 11, wherein step (ii) comprises
sequencing the DNA associated with the transcription factor; and wherein step (iii) comprises
using the presence of the transcription factor and the sequence of the associated DNA as a combined biomarker for determining a tissue affected by the disease in the subject.

14. The method of claim 13, wherein the tissue affected by the disease is the organ of origin.

15. The method of claim 11, wherein the disease is cancer or an inflammatory disease.

16. The method of claim 1, wherein the binding agent which binds to the transcription factor is an antibody or a fragment thereof.

17. The method of claim 1, wherein the body fluid sample is a blood, serum or plasma sample.

18. The method of claim 1, wherein the body fluid sample is a plasma sample which obtained by: (1) contacting a whole blood sample with a cross-linking agent; (2) contacting the cross-linked sample with a calcium ion chelating agent; and (3) isolating plasma from the sample. The method according to any one of claims 1 to 17, wherein the body fluid sample is a plasma sample which obtained by: (1) contacting a whole blood sample with a cross-linking agent; (2) contacting the cross-linked sample with a calcium ion chelating agent; and (3) isolating plasma from the sample.

19-22. (canceled)

23. A kit for the detection of a cell free chromatin fragment, comprising: a transcription factor and a DNA fragment as a combination biomarker which comprises a ligand or binder for the transcription factor optionally together with one or more of reagents for the amplification and/or sequencing of DNA associated with said transcription factor, a ligand or binder for nucleosomes, and instructions for use of the kit in accordance with the method of claim 1.

24. A method of treating cancer in a subject in need thereof, wherein said method comprises the following steps:
(a) contacting a body fluid sample obtained from the human or animal subject with a binding agent which binds to a transcription factor;
(b) detecting, measuring or sequencing a DNA fragment associated with the transcription factor; and
(c) using the presence, amount or sequence of DNA fragment as an indicator of the presence of cancer in the subject; and
(d) administering a treatment if the subject is determined to have cancer in step (c).

25-26. (canceled)


SEQUENCE LISTING (4 sequences)

SEQ ID NO: 1 - DNA, 9 bp, Homo sapiens
  misc_feature (3)..(3): n is a, c, g, or t
  misc_feature (6)..(7): n is a, c, g, or t
gcnctnnag

SEQ ID NO: 2 - DNA, 55 bp, Homo sapiens
gatcaagcacctggagggctcttcagagcaaagacaaacactgaggtcgctgcca

SEQ ID NO: 3 - DNA, 20 bp, Homo sapiens
tggccacacgagtgccctca

SEQ ID NO: 4 - DNA, 120 bp, Homo sapiens
cccaccccgttctgttcccccacagtttagacaagatcctcatgctccactggccacacgagtgccctcaggaggagtagacacaggtggagggagctccttttgaccagcagagaaaac